SEQUENCE LISTING (52 sequences)

SEQ ID NO: 1 - protein, 371 aa, Bacillus anthracis
MTERLKVMTIFGTRPEAIKMAPLVLELQKHPEKIESIVTVTAQHRQMLDQVLSIFGITPDFDLNIMKDRQTLIDITTRGLEGLDKVMKEAKPDIVLVHGDTTTTFIASLAAFYNQIPVGHVEAGLRTWDKYSPYPEEMNRQLTGVMADLHFSPTAKSATNLQKENKDESRIFITGNTAIDALKTTVKETYSHPVLEKLGNNRLVLMTAHRRENLGEPMRNMFRAIKRLVDKHEDVQVVYPVHMNPVVRETANDILGDYGRIHLIEPLDVIDFHNVAARSYLMLTDSGGVQEEAPSLGVPVLVLRDTTERPEGIEAGTLKLAGTDEETIFSLADELLSDKEAHDKMSKASNPYGDGRASERIVEAILKHFNK

SEQ ID NO: 2 - protein, 370 aa, Bacillus cereus
MTERLKVMTIFGTRPEAIKMAPLVLELQKHPEKIESIVTVTAQHRQMLDQVLSIFGITPDFDLNIMKDRQTLIDITTRGLEGLDKVMKEAKPDIVLVHGDTTTTFIASLAAFYNQIPVGHEAGLRTWDKYSPYPEEMNRQLTGVMADLHFSPTAKSATNLQKENKDESRIFITGNTAIDALKTTVKETYSHPVLEKLGNDRLVLMTAHRRENLGEPMRNMFRAIKRLVDKHEDVQVVYPVHMNPVVRETANDILGDHGRIHLIEPLDVIDFHNVAARSYLMLTDSGGVQEEAPSLGVPVLVLRDTTERPEGIEAGTLKLAGTDEETIFSLADELLSDKKAHDKMSKASNPYGDGRASERIVEAILKHFNK

SEQ ID NO: 3 - protein, 371 aa, Bacillus thuringiensis
MTERLKVMTIFGTRPEAIKMAPLVLELQKHPEKIESIVTVTAQHRQMLDQVLSIFGITPDFDLNIMKDRQTLIDITTRGLEGLDKVMKEAKPDIVLVHGDTTTTFIASLAAFYNQIPVGHVEAGLRTWDKYSPYPEEMNRQLTGVMADLHFSPTAKSATNLQKENKDESRIFITGNTAIDALKTTVKETYSHPVLEKLGNDRLVLMTAHRRENLGEPMRNMFRAIKRLVDKHEDVQVVYPVHMNPVVRETANDILGDHGRIHLIEPLDVIDFHNVAARSYLMLTDSGGVQEEAPSLGVPVLVLRDTTERPEGIEAGTLKLAGTDEETIFSLADELLSDKEAHDKMSKASNPYGDGRASERIVEAILKHFNK

SEQ ID NO: 4 - protein, 370 aa, Bacillus thuringiensis
MTERLKVMTIFGTRPEAIKMAPLVLELQKHPEKIESIVTVTAQHRQMLDQVLSIFGITPDFDLNIMKDRQTLIDITTRGLEGLDKVMKEAKPDIVLVHGDTTTTFIASLAAFYNQIPVGHVEAGLRTWDKYSPYPEEMNRQLTGVMADLHFSPTAKSATNLQKENKDESRIFITGNTAIALKTTVKETYSHPVLEKLGNDRLVLMTAHRRENLGEPMRNMFRAIKRLVDKHEDVQVVYPVHMNPVVRETANDILGDHGRIHLIEPLDVIDFHNVAARSYLMLTDSGGVQEEAPSLGVPVLVLRDTTERPEGIEAGTLKLAGTDEETIFSLADELLSDKEAHDKMSKASNPYGDGRASERIVEAILKHFNK

SEQ ID NO: 5 - protein, 374 aa, Bacillus cereus
MTERLKVMTIFGTRPEAIKMAPLVLELQKHPEKIESIVTVTAQHRQMLDQVLSIFGITPDFDLNIMKDRQTLIDITTRGLEGLDKVMKEAKPDIVLVHGDTTTTFIASLAAFYNQIPVGHDVEVEAGLRTWDKYSPYPEEMNRQLTGVMADLHFSPTAKSATNLQKENKDESRIFITGNTAIDALKTTVKETYSHPVLEKLGNDRLVLMTAHRRENLGEPMRNMFRAIKRLVDKHEDVQVVYPVHMNPVVRETANDILGDHGRIHLIEPLDVIDFHNVAARSYLMLTDSGGVQEEAPSLGVPVLVLRDTTERPEGIEAGTLKLAGTDEETIFSLADELLSDKEAHDKMSKASNPYGDGRASERIVEAILKHFNK

SEQ ID NO: 6 - protein, 371 aa, Bacillus cereus
MTERLKVMTIFGTRPEAIKMAPLVLELQKHPEKIESIVTVTAQHRQMLDQVLSIFGITPDFDLNIMKDRQTLIDITTRGLEGLDKVMKEAKPDIVLVHGDTTTTFIASLAAFYNQIPVGHVEAGLRTWDKYSPYPEEMNRQLTGVMADLHFSPTAKSATNLQKENKDESRIFITGNTAIDALQTTVKETYSHPVLEKLGNDRLVLMTAHRRENLGEPMRNMFRAIKRLVDKHEDVQVVYPVHMNPVVREIANDILGEHNRIHLIEPLDVIDFHNVAARSYLMLTDSGGVQEEAPSLGVPVLVLRDTTERPEGIEAGTLKLAGTDEETIFGLADELLSDKEAHDKMAKASNPYGDGRASERIVEAILQHFNK

SEQ ID NO: 7 - protein, 377 aa, Enterococcus faecium
MKIKIMTIFGTRPEAIKMAPLIKAIENDERFESIVTVTAQHRQMLDQVMDIFDLKADYDLNIMKDGQTLTDVTSRVIKELDSVLVEAKPDIILVHGDTTTTFAASIAGFYHQIKIGHVEAGLRTWNKYSPFPEEMNRQLTDTLADIYFAPTVMSKSNLLKEGRSEKSIFITGNTAIDAMKYTIKQNYSNDLLDNLAGKRIILVTMHRRENLGQPMTNVFKAINRLIEKFEDVHIVFPMHKNPKVRKNAEETFNDSEQVHLIEPLDVIDFQNFSNNSYMILSDSGGVQEEAPSLGVPVLVLRDTTERPEGIEVGTLKLVGTEEDKVFEEATLLLSDKEEYKKMSQASNPYGDGNASERILDAIAYNFGIQQEKPIDFQ

SEQ ID NO: 8 - protein, 382 aa, Enterococcus faecalis
MMKKIKVMTVFGTRPEAIKMAPLIKVLEEQSEGFDSVVVVTAQHRQMLDQVLEDFQITPNHDLNIMKDGQTLTDITSRVLNLLTEVLVTEQPDIVLVHGDTTTSFAAALAAFYQQIPVGHVEAGLRTWQKYSPFPEEMNRQLVDVLTDIYFAPTTQSKGNLIKENHPEEHIYVTGNTAIDAMAYTVDAHYQNDLLEKIPTDQRIVLITMHRRENLGLPMANVFKAVRRLVMEHPEIEVIFPMHKNPKVREIVAEHLGELARVHLIEPLDVKDFQNFAAKSSLILTDSGGVQEEAPSLGVPVLVLRDTTERPEGVAAGTLKLVGTDEQVYQEAKTLLTDSDAYHAMAHAQNPYGDGQASHRIVEAIAYEMQQSDKKPDTFTAK

SEQ ID NO: 9 - protein, 391 aa, Staphylococcus aureus
MCLNFREDNVMKKIMVIFGTRPEAIKMAPLVKEIDHNGNFEANIVITAQHRDMLDSVLSIFDIQADHDLNIMQDQQTLAGLTANALAKLDSIINEEQPDMILVHGDTTTTFVGSLAAFYHQIPVGHVEAGLRTHQKYSPFPEELNRVMVSNIAELNFAPTVIAAKNLLFENKDKERIFITGNTVIDALSTTVQNDFVSTIINKHKGKKVVLLTAHRRENIGEPMHQIFKAVRDLADEYKDVVFIYPMHKNPKVRAIAEKYLSGRNRIELIEPLDAIEFHNFTNQSYLVLTDSGGIQEEAPTFGKPVLVLRNHTERPEGVEAGTSRVIGTDYDNIVRNVKQLIEDDEAYQRMSQANNPYGDGQASRRICEAIEYYFGLRTDKPDEFVPLRHK

SEQ ID NO: 10 - protein, 311 aa, Bacillus anthracis
FDLNIMKDRQTLIDITTRGLEGLDKVMKEAKPDIVLVHGDTTTTFIASLAAFYNQIPVGHVEAGLRTWDKYSPYPEEMNRQLTGVMADLHFSPTAKSATNLQKENKDESRIFITGNTAIDALKTTVKETYSHPVLEKLGNNRLVLMTAHRRENLGEPMRNMFRAIKRLVDKHEDVQVVYPVHMNPVVRETANDILGDYGRIHLIEPLDVIDFHNVAARSYLMLTDSGGVQEEAPSLGVPVLVLRDTTERPEGIEAGTLKLAGTDEETIFSLADELLSDKEAHDKMSKASNPYGDGRASERIVEAILKHFNK

SEQ ID NO: 11 - protein, 311 aa, Bacillus anthracis
FDLNIMKDRQTLIDITTRGLEGLDKVMKEAKPDIVLVHGDTTTTFIASLAAFYNQIPVGHVEAGLRTWDKYSPYPEEMNRQLTGVMADLHFSPTAKSATNLQKENKDESRIFITGNTAIDALKTTVKETYSHPVLEKLGNDRLVLMTAHRRENLGEPMRNMFRAIKRLVDKHEDVQVVYPVHMNPVVRETANDILGDHGRIHLIEPLDVIDFHNVAARSYLMLTDSGGVQEEAPSLGVPALVLRDTTERPEGIEAGTLKLAGTDEETIFSLADELLSDKEAHDKMSKASNPYGDGRASERIVEAILKHFNK

SEQ ID NO: 12 - protein, 371 aa, Bacillus anthracis
MTERLKVMTIFGTRPEAIKMAPLVLELQKHPEKIESIVTVTAQHRQMLDQVLSIFGITPDFDLNIMKDRQTLIDITTRGLEGLDKVMKEAKPDIVLVHGDTTTTFIASLAAFYNQIPVGHVEAGLRTWDKYSPYPEEMNRQLTGVMADLHFSPTAKSATNLQKENKDESRIFITGNTAIDALKTTVKETYSHPVLEKLGNDRLVLMTAHRRENLGEPMRNMFRAIKRLVDKHEDVQVVYPVHMNPVVRETANDILGDHGRIHLIEPLDVIDFHNVAARSYLMLTDSGGVQEEAPSLGVPALVLRDTTERPEGIEAGTLKLAGTDEETIFSLADELLSDKEAHDKMSKASNPYGDGRASERIVEAILKHFNK

SEQ ID NO: 13 - protein, 365 aa, Streptococcus pneumoniae
MKKVVVVFGTRPEAIKMCPLVKELRTRKNIETLVCVTGQHRQMLDQVLDTFGIIPDFDLSIMKDKQTLFDVTIGILEGMKAILESEKPDLVLVHGDTSTTFASSLAAFYLQIPIGHVEAGLRTYDIYSPYPEEFNRQAVGVLAQYHFTPTQLSKDNLLREGKTPESIFVTGNTAIDALQTTIQEDYTHPELEWIGESRFILITAHRRENLGEPMRHMFRAIRRIIEEYSDVKAIYPIHMNPRVRQIAEEELSGCERIKMIEPLEVLDFHNFLSRSYLILTDSGGIQEEAPSLGKPVLVMRDTTERPEGIEAGTLKLVGADENNIYRHFKELLENDSVYQAMSQASNPYGDGTACKKIADILEGEV

SEQ ID NO: 14 - protein, 382 aa, Streptococcus mutans
MKKIKVMLVFGTRPEAIKMAPLIISLKEQTERFETVTVVTAQHRQMLDQVLETFKIRPDYDLNIMSKQQTLSTITTNVINKLDEVLKSEKPDIILVHGDTTTTLAASISAFYNQIKIGHVEAGLRTWNKYSPFPEEVNRQITDVVSDLYFAPTNQSRDNLLKENHPAQHIFITGNTAIDALDLTVKENYHHDVLTKIKSDNRIILVTMHRRENQGEPMRRVFKTLKSVLADYPDVELVYPVHLSPAVQKAAKDILANTERIHLIEPLDVMDFHNLANKSYFIMSDSGGVQEEAPSLGKPVLVLRDTTERPEGVAAGTLRLVGTQEDSVKNAMISLLDNKEEYDKMAQTQNPYGDGQASKRILEAISYYFNNGARPDDFGTGE

SEQ ID NO: 15 - protein, 377 aa, Enterococcus faecium; identical to SEQ ID NO: 7

SEQ ID NO: 16 - protein, 383 aa, Enterococcus faecalis
MMKKIKVMTVFGTRPEAIKMAPLIKVLEEQSEGFDSVVVVTAQHRQMLDQVLEDFQITPNHDLNIMKDGQTLTDITSRVLNLLTEVLVTEQPDIVLVHGDTTTSFAAALAAFYQQIPVGHVEAGLRTWQKYSPFPEEMNRQLVDVLTDIYFAPTTQSKGNLIKENHPEEHIYVTGNTAIDAMAYTVDAHYQNDLLEKIPTDQRIVLITMHRRENLGLPMANVFKAVRRLVMEHPEIEVIFPMHKNPKVREIVAEHLGELARVHLIEPLDVKDFQNFAAKSSLILTDSGGVQEEAPSLGVPVLVLRDTTERPEGVAAGTLKLVGTDEQVVYQEAKTLLTDSDAYHAMAHAQNPYGDGQASHRIVEAIAYEMQQSDKKPDTFTAK

SEQ ID NO: 17 - protein, 376 aa, Staphylococcus aureus
MMKKIMTIFGTRPEAIKMAPLVKALEQEKMLEPIVVVTAQHREMLDSVLSTFEIKPKYDLNIMKSGQTLSEITSKSITQLEQVIQLEKPDMVLVHGDTMTTFAGGLAAFYNQVPIGHVEAGLRSYDKYSPFPEEVNRQLVGVLADLHFAPTKNAASHLLSEGKYSESVVVTGNTAIDAMKYTVDDNYKSNIMDKYHDKKFILMTAHRRENIGKPMENIFKAVRRLIDEYTDLALVYPMHKNPKVREVAQKILGSHDRIELIEPLDVVDFHNFAKKSYFILTDSGGIQEEAPSFNKPVLVLRSVTERPEGVEAGTLKVIGTNKQNVYQAAKELIDDERLYHQMSEASNPYGDGFASERIVNHIKYYLNLITEKPSDF

SEQ ID NO: 18 - protein, 376 aa, Staphylococcus aureus; identical to SEQ ID NO: 17

SEQ ID NO: 19 - protein, 375 aa, Staphylococcus aureus
MKKIMTIFGTRPEAIKMAPLVKALEQEKMLEPIVVVTAQHREMLDSVLSTFEIKPKYDLNIMKSGQTLSEITSKSITQLEQVIQLEKPDMVLVHGDTMTTFAGGLAAFYNQVPIGHVEAGLRSYDKYSPFPEEVNRQLVGVLADLHFAPTKNAASHLLSEGKYSESVVVTGNTAIDAMKYTVDDNYKSNIMDKYHDKKFILMTAHRRENIGKPMENIFKAVRRLIDEYTDLALVYPMHKNPKVREVAQKILGSHDRIELIEPLDVVDFHNFAKKSYFILTDSGGIQEEAPSFNKPVLVLRSVTERPEGVEAGTLKVIGTNKQNVYQAAKELIDDERLYHQMSEASNPYGDGFASERIVNHIKYYLNLITEKPSDF

SEQ ID NO: 20 - protein, 379 aa, Listeria monocytogenes
MAKIKVMSIFGTRPEAIKMAPLVLALEKEPETFESTVVITAQHREMLDQVLEIFDIKPDIDLDIMKKGQTLAEITSRVMNGINEVIAAENPDIVLVHGDTTTSFAAGLATFYQQKMLGHVEAGLRTWNKYSPFPEEMNRQLTGVMADIHFSPTKQAKENLLAEGKDPATIFVTGNTAIDALKTTVQKDYHHPILENLGDNRLILMTAHRRENLGEPMQGMFEAVREIVESREDTELVYPMHLNPAVREKAMAILGGHERIHLIEPLDAIDFHNFLRKSYLVFTDSGGVQEEAPGMGVPVLVLRDTTERPEGIEAGTLKLIGTNKENLIKEALDLLDNKESHDKMAQAANPYGDGFAANRILAAIKSHFEETDRPEDFIV

SEQ ID NO: 21 - protein, 391 aa, Staphylococcus aureus
MCLNFREDNVMKKIMVIFGTRPEAIKMAPLVKEIDHNGNFEANIVITAQHRDMLDSVLSIFDIQADHDLNIMQDQQTLAGLTANALAKLDSIINEEQPDMILVHGDTTTTFVGSLAAFYHQIPVGHVEAGLRTHQKYSPFPEELNRVMVSNIAELNFAPTVIAAKNLLFENKDKERIFITGNTVIDALSTTVQNDFVSTIINKHKGKKVVLLTAHRRENIGEPMHQIFKAVRDLADEYKDVVFIYPMHRNPKVRAIAEKYLSGRNRIELIEPLDAIEFHNFTNQSYLVLTDSGGIQEEAPTFGKPVLVLRNHTERPEGVEAGTSRVIGTDYDNIVRNVKQLIEDDEAYQRMSQANNPYGDGQASRRICEAIEYYFGLRTDKPDEFVPLRHK

SEQ ID NO: 22 - protein, 381 aa, Staphylococcus aureus
MKKIMVIFGTRPEAIKMAPLVKEIDHNGNFEANIVITAQHRDMLDSVLSIFDIQADHDLNIMQDQQTLAGLTANALAKLDSIINEEQPDMILVHGDTTTTFVGSLAAFYHQIPVGHVEAGLRTHQKYSPFPEELNRVMVSNIAELNFAPTVIAAKNLLFENKDKERIFITGNTVIDALSTTVQNDFVSTIINKHKGKKVILLTAHRRENIGEPMHQIFKAVRDLADEYKDVVFIYPMHRNPKVRAIAEKYLSGRNRIELIEPLDAIEFHNFTNQSYLVLTDSGGIQEEAPTFGKPVLVLRNHTERPEGVEAGTSRVIGTDYDNIVRNVKQLIEDDEAYQRMSQANNPYGDGQASRRICEAIEYYFGLRTDKPDEFVPLRHK

SEQ ID NO: 23 - protein, 381 aa, Staphylococcus aureus
MKKIMVIFGTRPEAIKMAPLVKEIDHNGNFEANIVITAQHRDMLDSVLSIFDIQADHDLNIMQDQQTLADLTANALAKLDSIINEEQPDMILVHGDTTTTFVGSLAAFYHQIPVGHVEAGLRTHQKYSPFPEELNRVMVSNIAELNFAPTVIAAKNLLFENKDKERIFITGNTVIDALSTTVQNDFVSTIINKHKGKKVILLTAHRRENIGEPMHQIFKAVRDLADEYKDVVFIYPMHRNPKVRAIAEKYLSGRNRIELIEPLDAIEFHNFTNQSYLVLTDSGGIQEEAPTFGKPVLVLRNHTERPEGVEAGTSRVIGTDYDNIVRNVKQLIEDDEAYQRMSQANNPYGDGQASRRICEAIEYYFGLRSDKPDEFVPLRHK

SEQ ID NO: 24 - protein, 394 aa, Streptococcus pneumoniae
MKIKTDYSDIHFKDNGKLKLLIIVGTRPEIIRLSSVITKCRKYFDVILAHTGQNYDYNLNGIFFDNLGLDTPDVYMDAVGDDLGATVGNIINTSYKLMNQIKPDALLILGDTNSCLSAIAAKRLHIPIFHMEAGNRCKDECLPEETNRRIVDVISDVNLAYSEHARKYLHECGLPKERTYVTGSPMAEVLHKNLSAIESSDIHERLGLKKGGYILLSAHREENIDTDKNFISLFTAINQLAEKYNMPILYSCHPRSKKRLQESGFKLDKRVIQHEPLGFHDYNCLQMNAFVVVSDSGTLPEESSFFTSQGYPFPAVCIRTSTERPESLDKAGFILAGIDENSLLQAVETAVSLAQDEDFGLPVPDYVEENVSTKVVKIIQSYTGIVDKIVWRKS

SEQ ID NO: 25 - protein, 374 aa, Staphylococcus aureus
MEKLKLMTIVGTRPEIIRLSSTIKACDQYFNQILVHTGQNYDYTLNQIFFDDLELRQPDHYLEAVGSNLGETMGNIIAKTYDVLLREQPDALLILGDTNSCLAAVSAKRLKIPVFHMEAGNRCFDQNVPEEINRKIVDHVSDVNLPYTEHSRRYLLDEGFNKANIFVTGSPMTEVIEAHRDKINHSDVLNKLGLEPQQYILVSAHREENIDNEKNFKSLMNAINDIAKKYKMPVIYSTHPRSWKKIEESKFEFDPLVKQLKPFGFFDYNALQKDAFVVLSDSGTLSEESSILKFPGVLIRTSTERPEVLDKGTVIVGGITYNNLIQSVELAREMQNNNEPMIDAIDYKDTNVSTKVVKIIQSYKDIINRNTWRK

SEQ ID NO: 26 - protein, 374 aa, Staphylococcus aureus
MEKLKLMTIVGTRPEIIRLSSTIKACDQYFNQILVHTGQNYDYTLNQIFFDDLELRQPDHYLEAVGSNLGETMGNIIAKTYDVLLREQPDALLILGDTNSCLAAVSAKRLKIPVFHMEAGNRCFDQNVPEEINRKIVDHVSDVNLPYTEHSRRYLLDEGFNKANIFVTGSPMTEVIEAHRDKINHSDVLNKLGLEPQQYILVSAHREENIDNEKNFKSLMNAINDIAKKYKMPVIYSTHPRSWKKIEESKFEFDPLVKKLKPFGFFDYNALQKDAFVVLSDSGTLSEESSILKFPGVLIRTSTERPEVLDKGTVIVGGITYNNLIQSVELAREMQNNNEPMIDAIDYKDTNVSTKVVKIIQSYKDIINRNTWRK

SEQ ID NO: 27 - protein, 374 aa, Staphylococcus aureus; identical to SEQ ID NO: 25

SEQ ID NO: 28 - protein, 374 aa, Enterococcus faecium
MKKLKVMTVVGTRPEIIRLSAVINRLDQSEAIEHILVHTGQNYDYELNEVFFEDFKLKKPDYFLNAATGTAIETVGNILIKIDPILEEVKPDAFLVLGDTNSCLCAIAAKRRHIPIFHMEAGNRCFDQRVPEETNRKIVDHTADINLTYSDIAREYLLREGLPADRVIKTGSPMFEVLNSRKDDIQHSDILNRLVLKEGQYFVVSAHREENISSETNFLNLVDSLNTIAETYQLPIIISTHPRTMKMIEAKGIKFHELIQTMKPMGFNDYNKLQINAKAVLSDSGTISEESSILNFKALNIRQAHERPEAMEEASVMMVGLEKERIMQGLEVLETQQKDTLGHVADYSMPNVSEKVLRIILSYTDYVNRVVWGR

SEQ ID NO: 29 - protein, 377 aa, Streptococcus suis
MKKICFVTGSRAEYGIMRRLLSYLQDDPEMELDLVVTAMHLEEKYGMTVKDIEADKRRIVKRIPLHLTDTSKQTIVKSLATLTEQLTVLFEEVQYDLVLILGDRYEMLPVANAALLYNIPICHIHGGEKTMGNFDESIRHAITKMSHLHLTSTDEFRNRVIQLGENPNHVLNIGAMGVENVLKQDFLTREELAMELGIDFAEDYYVVLFHPVTLEDNTAEEQTQALLDALKEDGSQCLIIGSNSDTHADKIMELMHEFVKQDSDSYIFTSLPTRYYHSLVKHSQGLIGNSSSGLIEVPSLQVPTLNIGNRQFGRLSGPSVVHVGTSKEAIVGGLGQLRDVIDFTNPFEQPDSALQGYRAIKEFLSVQASTMKEFYDR

SEQ ID NO: 30 - protein, 384 aa, Streptococcus agalactiae
MKKICLVTGSRAEYGIMKPLIQRLSKDKEVNLQIIATAMHLEEKYGYTYRQIEEDGFDIAYKVPLHLYDTDRRTVSTAMAHLQLGLTKIFDKEDYDLVIILGDRYEMLPVVNVALIYNVPVCHLHGGETSLGNFDEYIRHAVTKMSHLHLVSTEDFRQRVIQMGEQPQFVINTGALGVENALSIPSLTKEAIEKQLGIVLEESYFVVLYHPVTFEQGKSAGEQMKAVLSALSKFGVQCLFIGSNSDTGSDDIAKAINTYLINHENSYCFASLSTQLYHSLIRHSLGLIGNSSSGLIEVPSLMKPTLNIGDRQKGRLHGESVVSVPVETSSVLEGLSKLNEVTNFDNPYYKGNASSIAYEAIKLYLKDEPSISQPFYDLKENNLK

SEQ ID NO: 31 - protein, 376 aa, Escherichia coli
  misc_feature (1)..(1): Xaa can be any naturally occurring amino acid
XKVLTVFGTRPEAIKXAPLVHALAKDPFFEAKVCVTAQHREXLDQVLKLFSIVPDYDLNIXQPGQGLTEITCRILEGLKPILAEFKPDVVLVHGDTTTTLATSLAAFYQRIPVGHVEAGLRTGDLYSPWPEEANRTLTGHLAXYHFSPTETSRQNLLRENVADSRIFITGNTVIDALLWVRDQVXSSDKLRSELAANYPFIDPDKKXILVTGHRRESFGRGFEEICHALADIATTHQDIQIVYPVHLNPNVREPVNRILGHVKNVILIDPQEYLPFVWLXNHAWLILTDSGGIQEEAPSLGKPVLVXRDTTERPEAVTAGTVRLVGTDKQRIVEEVTRLLKDENEYQAXSRAHNPYGDGQACSRILEALKNNRISL

SEQ ID NO: 32 - protein, 376 aa, Klebsiella pneumoniae
MKVLTVFGTRPEAIKMAPLVHALAKDPHFEAKVCVTAQHREMLDQVLKLFSIVPDYDLNIMKPGQGLTEITCRILEGLKPVLESFKPDVVLVHGDTTTTMAASLAAFYQRIPVGHVEAGLRTGDLSSPWPEEGNRTLTGHLATYHFAPTETSRQNLLRENIADSRITVTGNTVIDALFWVRDRVLGDEALRETLLQRYPFISHGKKMILVTGHRRESFGLGFEQICQALAEIAHTHPEVQIVYPVHLNPNVSEPVNRILGHIDNVMLIEPQDYLPFVWLMDRAWLILTDSGGIQEEAPSLGKPVLVMRDMTERPEAVAAGTVCLVGTDSQRIVAEVTRLLQDDAAYQAMSRAHNPYGDGEACRRILSALKNNQVTL

SEQ ID NO: 33 - protein, 411 aa, Pseudomonas syringae
MKVLSIFGTRPEAIKMAPLVRALAAEPGIDSRICITGQHQSMLQQVLDMFELKADYSLDVMRPDQTLNSLTAALYAAIDPILDEMKPDKVLVHGDTTSAMVAAMSAFHRRIPIGHVEAGLRTGDIRQPWPEEMNRRCIDLISDHLFAPTAESRRNVLGERLQGISFVTGNTVIDALHLTAQRIDSNRQLRHALDRQFSFLVPERKVLVVTGHRRENFGDGFLNICKALGELARRDDIQIVYPVHLNPNVLGPVTEHLGDLPNVHLIKPLDYLSFVRLMQRAHVILTDSGGVQEEAPSLGKPVLVMRDVTERPEAVAAGTVRLVGTETDAIIRGVNALFDDDALWQRASHAANPYGDGKASARIVDALMGRPVDEFVAELPRYRTDPVDVQLDTLIQPQLQHQHAQMRSMAS

SEQ ID NO: 34 - protein, 354 aa, Pseudomonas aeruginosa
MKILTIIGARPQFIKASVVSKAIIEQQTLSEIIVHTGQHFDANMSEIFFEQLGIPKPDYQLDIHGGTHGQMTGRMLMEIEDVILKEKPHRVLVYGDTNSTLAGALAASKLHVPIAHIEAGLRSFNMRMPEEINRILTDQVSDILFCPTRVAIDNLKNEGFERKAAKIVNVGDVMQDSALFFAQRATSPIGLASQDGFILATLHRAENTDDPVRLTSIVEALNEIQINVAPVVLPLHPRTRGVIERLGLKLEVQVIDPVGYLEMIWLLQRSGLVLTDSGGVQKEAFFFGKPCVTMRDQTEWVELVTCGANVLVGAARDMIVESARTSLGKTIQDDGQLYGGGQASSRIAEYLAKL

SEQ ID NO: 35 - protein, 372 aa, Neisseria meningitidis
MKVLTVFGTRPEAIKMAPVILELQKHNTITSKVCITAQHREMLDQVLSLFEIKADYDLNIMKPNQSLQEITTNIISSLTDVLEDFKPDCVLVHGDTTTTFAASLAAFYQKIPVGHIEAGLRTYNLYSPWPEEANRRLTSVLSQWHFAPTEDSKNNLLSESIPSDKVIVTGNTVIDALMVSLEKLKITTIKKQMEQAFPFIQDNSKVILITAHRRENHGEGIKNIGLSILELAKKYPTFSFVIPLHLNPNVRKPIQDLLSSVHNVHLIEPQEYLPFVYLMSKSHIILSDSGGIQEEAPSLGKPVLVLRDTTERPEAVAAGTVKLVGSETQNIIESFTQLIEYPEYYEKMANIENPYGIGNASKIIVETLLKNR

SEQ ID NO: 36 - protein, 378 aa, Acinetobacter baumannii
MKKIFISIVFGTRPELIKLAPVILLAKQDSRFQVEVIFTGQHDELVRDAIDFFGVEIDHRLKIMNAGQSLNQLLIHGLTQLENIYTDGQKRDAIVIQGDTTTVLAAGLVAFSMKIPVAHVEAGLRSYDLDHPFPEEGNRQLVSRITKWHFAPTEQSKRNLLNEQIPPSLITVTGNTVVDAVYLGRKLIAEKTGLKNQLEPYGIELKQNDKVVLITAHRRENFGEGIQNICNAVEYLAKHHPDLHFIWPVHLNPAVHNVVHDKFKNHAQIHLVKPLDYPSLLAVIDRSTFILTDSGGLQEESPSFNKPVLILRDTTERPEVVEVGAGVLVGTNQQKIIEEAEKLLTDSQHYQKMAHVENPFGDGCAAQRILDEIARTYN

SEQ ID NO: 37 - DNA, 15 bp, Artificial Sequence
  Primer
ccgtcgtgaaaactt

SEQ ID NO: 38 - DNA, 15 bp, Artificial Sequence
  Primer
ccgtcgtgaaaactt

SEQ ID NO: 39 - DNA, 20 bp, Artificial Sequence
  Primer
taatggcggaccttcatttc

SEQ ID NO: 40 - DNA, 22 bp, Artificial Sequence
  Primer
caagaaccggtacaccaagtga

SEQ ID NO: 41 - DNA, 28 bp, Artificial Sequence
  Primer
gttggaattgtaggtttaaatggttctg

SEQ ID NO: 42 - DNA, 26 bp, Artificial Sequence
  Primer
ggaacagtggatattaaaggttcagc

SEQ ID NO: 43 - DNA, 23 bp, Artificial Sequence
  Primer
ccagtacatggcgttccttactt

SEQ ID NO: 44 - DNA, 23 bp, Artificial Sequence
  Primer
agagctccgcgatatacttctac

SEQ ID NO: 45 - DNA, 62 bp, Artificial Sequence
  Primer
ggggacaagtttgtacaaaaaagcaggctcatgtataataatacagtaacaatactaccaga

SEQ ID NO: 46 - DNA, 50 bp, Artificial Sequence
  Primer
ggggaccactttgtacaagaaagctgggtgaaggtccgccattacgcctg

SEQ ID NO: 47 - DNA, 31 bp, Artificial Sequence
  Primer
gtaggtaccggcacctcttgtattagagttg

SEQ ID NO: 48 - DNA, 32 bp, Artificial Sequence
  Primer
gtaggtacccaacacgaggtttagaaggtttg

SEQ ID NO: 49 - DNA, 31 bp, Artificial Sequence
  Primer
cgtactagagaaacttggaaataatcgtctt

SEQ ID NO: 50 - DNA, 23 bp, Artificial Sequence
  Primer
gcacggaacatattacgcattgg

SEQ ID NO: 51 - DNA, 29 bp, Artificial Sequence
  Primer
agctgaaacattagtagatccagaaactg

SEQ ID NO: 52 - DNA, 23 bp, Artificial Sequence
  Primer
aatgcgatcaagtgtacgacgat

(19) United States
(12) Patent Application Publication
(10) Pub. No.: US 2014/0073639 A1
(43) Pub. Date: Mar. 13, 2014

(54) INHIBITION OF ANTIMICROBIAL TARGETS WITH REDUCED POTENTIAL FOR RESISTANCE

(71) Applicants: The Rockefeller University, New York, NY (US); Avacyn Pharmaceuticals, Inc., Teaneck, NJ (US)

(72) Inventors: Vincent A. FISCHETTI, New York, NY (US); Allan R. GOLDBERG, Teaneck, NJ (US); Raymond SCHUCH, New York, NY (US)

(73) Assignees: The Rockefeller University, New York, NY (US); Avacyn Pharmaceuticals, Inc., Teaneck, NJ (US)

(21) Appl. No.: 13/962,623

(22) Filed: Aug. 8, 2013

Related U.S. Application Data
(60) Provisional application No. 61/680,791, filed on Aug. 8, 2012.

(51) Int. Cl. C07D 513/04; C07D 409/06; C07D 235/28; C07D 417/14; C07D 417/04; C07D 417/06; C07D 405/06; C07D 239/66; C07D 403/04; C07D 233/96; C07D 405/14; C07D 403/06; C12Q 1/533; C07D 413/12

(52) U.S. Cl. CPC C07D 513/04; C12Q 1/533; C07D 409/06; C07D 235/28; C07D 417/14; C07D 417/04; C07D 413/12; C07D 405/06; C07D 239/66; C07D 403/04; C07D 233/96; C07D 405/14; C07D 403/06; C07D 417/06
USPC 514/234.2; 435/7.8; 514/270; 514/234.5; 514/369; 514/364; 514/389; 514/341; 514/378; 514/235.8

(57) ABSTRACT

The application describes targets and methods that can inhibit bacterial growth in Gram-positive and Gram-negative bacteria. A bacterial enzyme, 2-epimerase, is common to both Gram-positive and Gram-negative bacteria and contains an allosteric site that can be targeted to disrupt the enzyme. The allosteric site is present on the bacterial 2-epimerase, but the analogous mammalian enzyme does not contain the allosteric site, providing a route for attacking bacterial infections without affecting the mammalian enzyme.

### CROSS-REFERENCE TO RELATED APPLICATIONS

This application claims the benefit of U.S. Provisional Patent Application No. 61/680,791, filed 8 Aug. 2012, and incorporated herein by reference in its entirety as if fully set forth below.

The instant application contains a Sequence Listing which has been submitted in ASCII format via EFS-Web and is hereby incorporated by reference in its entirety. Said ASCII copy, created on Aug. 8, 2013, is named avacyn_ST25.txt and is 119 kbytes in size.

### TECHNICAL FIELD

The invention relates to the discovery of antimicrobial targets with reduced potential for resistance and to inhibitors of those targets. The targets are enzymes that are essential for the survival of bacteria, particularly infectious bacteria. The inhibitors interfere with or disable a target enzyme, for example by acting as ligands that bind to the enzyme and prevent its essential function, causing the bacteria to die. These discoveries provide for antibacterial drugs comprising an inhibitor that are useful for treating bacterial infections caused by Gram-positive and Gram-negative bacteria. These antibacterial drugs, and pharmaceutical compositions and formulations comprising them, are effective drugs against bacteria that are resistant to other antimicrobial drugs. Further, the drugs of the invention tend to not induce bacteria to develop evolutionary resistance.

Suitable target enzymes of the invention are essential cell wall biosynthetic enzymes which are needed for bacterial growth. These bacterial enzymes can be identified through an indirect methodology using lysins, which are enzymes expressed by viruses (bacteriophages) that infect bacteria, and have binding characteristics that recognize critical receptors within bacterial cell walls. (1) One critical bacterial enzyme in a pathway identified through the use of bacteriophage lysin experiments is called UDP-N-acetylglucosamine-2-epimerase (2-epimerase). This enzyme can be inhibited by small molecule compositions, including the compound 2-{4-[5-(4-bromophenyl)-thiophen-2-ylmethylene]-5-oxo-2-thioxo-imidazolidin-1-yl}-3-phenyl-propionic acid, called Epimerox (33). The 2-epimerase target and Epimerox inhibitor are representative of the antimicrobial targets, inhibitors, pharmaceutical compositions, and method of treatment of the invention.

### BACKGROUND

Both antibiotic drugs and synthetic antibacterial drugs inhibit the growth of bacteria, or destroy bacteria and other microorganisms, and are used for the treatment of infectious diseases. Over time, and with increasing use of a drug in a population, microorganisms can adapt or evolve to develop resistance to the drug. The drug might become less effective or even ineffective as a treatment for disease, and other drugs that are still effective might not be available. Thus, a resistant microorganism is able to survive exposure to an antibiotic. Drug resistance is an increasing problem in medicine. There is a growing need for antimicrobial drugs that remain effective and have less potential to induce resistance.

Resistant microorganisms can emerge through natural selection, from a population of microorganisms that are not resistant, because of spontaneous genetic mutations or mutations that are induced, for example by environmental factors. Genes that confer resistance are encoded in the DNA of one or more bacteria, particularly those with a common ancestry, and can be activated because of evolutionary pressure. Resistance genes can be transferred from one bacterium to another of the same type or different bacterial species through natural processes, e.g. horizontal gene transfer via transposable genetic elements. Thus, a gene for antibiotic resistance that evolves or emerges via natural selection may be disseminated throughout a diverse population of microorganisms. Exposure to antibiotics, as used in modern medicine, can be an evolutionary stress that selects for genes which express the antibiotic resistance trait. Genes or their expression products, such as proteins or enzymes, which are essential for bacterial growth, but do not have resistant counterparts or mutations, or which are slow or unable to take on resistant forms, would be ideal targets for drug intervention. Antibiotic use can increase selective pressure in a population of bacteria allowing resistant bacteria to thrive and causing susceptible bacteria to die. As resistance becomes more common, a greater need for alternative treatments arises. Antibiotic resistance to many different types of antibacterial drugs already is a significant public health problem.

The long-term and large-scale use of antibiotics in human and veterinary medicine in particular provides a powerful selective pressure for antibiotic-resistance to arise and eventually dominate populations of human pathogenic microorganisms. (2) Spontaneous resistance to most antibiotics appears with frequencies that generally range from ≦10⁻⁸ to 10⁻⁹ and, through a series of successive mutations, ultimately generates clinically significant resistance. Such resistance then can be propagated or mobilized in an intra- and inter-species manner by genetic elements, including transposons, plasmids, integrons and genomic islands. (3) The evolution of multidrug resistance and the international dissemination of epidemic clones exacerbates the problem, highlighting the need for new antimicrobial development strategies that address the issue of evolving resistance.

A novel class of antimicrobial agents was recently identified, called lysins, which are notable in several cases for their species specificity and the lack of bacterial resistance to their activity. (1, 4) Lysin enzymes are bacteriophage-encoded cell wall hydrolases, required by bacteriophage during the late phase of infection of bacteria. Lysins function to hydrolyze or cleave certain chemical bonds of peptidoglycans (a structural component of the bacteria's cell wall), lyse (destroy by breaking open) the bacterial host, and release progeny virions. Purified lysins also can be potent lytic agents outside the viral context, driving lysis “from without” of target bacteria both in vitro and in experimentally-infected animals. (4-7) Therapeutic lysins generally have modular structures defined by well-conserved N-terminal peptidoglycan-cleaving domains and more divergent C-terminal cell wall binding (CBD) domains that can recognize species-specific cell wall glycopolymers (CWGs). The largely universal nature of lysin-sensitive cleavage sites in peptidoglycan, combined with an increasing understanding of roles for CWGs in maintaining cell wall integrity, is cited to explain the absence of resistance to certain lysins. (1)

Although certain lysins themselves are promising as candidates for resistance-improved or resistance-free antibiotics, they also have significant disadvantages, particularly with regard to their pharmacokinetic properties. Lysins, like other foreign proteins delivered systemically to animals, are quickly degraded. Thus, if lysins were to be used systemically, they would need to be modified to extend their half-life, or they would need to be delivered frequently by IV infusion. An additional concern for the use of lysins is the development of neutralizing antibodies that can reduce their in vivo effectiveness during treatment. Unlike antibiotics, which are small molecules that are not generally immunogenic, enzymes are proteins that are capable of stimulating an immune response, which would interfere with lysin activity in vivo. Thus, there remains a need for additional antimicrobial targets and corresponding antimicrobial drug agents, particularly small molecules, which are safe, efficacious, robust, and do not stimulate resistance. The biosynthetic pathways of bacteria that are affected by lysins are one potential source for new targets and new therapeutic interventions.

### SUMMARY OF THE INVENTION

The invention provides compositions and methods to identify and inhibit antimicrobial targets having reduced potential for the development of resistance, leading to pharmaceutical compositions, methods of treatment, and methods of making and using such compositions and treatments. The invention includes antimicrobial agents with reduced potential for induction of drug resistance, and methods for discovering, designing, making and using such antimicrobial agents.

Various exemplary embodiments herein provide for methods of identifying antimicrobial targets by use of viral proteins, particularly bacteriophage proteins, which interact with specific antimicrobial targets. Various exemplary embodiments herein also provide for methods of treating a bacterial infection by targeting the antimicrobial targets. Further, exemplary embodiments provide for methods of treating bacterial infection by targeting 2-epimerase or a variant or relative thereof. The 2-epimerase enzyme may be found within a Gram-positive or Gram-negative bacteria. A preferred method is to treat an infection caused by the Gram-positive bacteria Bacillus anthracis by targeting 2-epimerase. One way to target 2-epimerase, and a preferred embodiment of the invention, is to inhibit its essential function in the life-cycle of a bacteria by introducing the enzyme to a small molecule that binds to, and incapacitates, the enzyme, e.g. by blocking its active site or interacting with an allosteric site, thus altering the conformation of the enzyme or its active site such that its function is lost or impaired. Compounds suitable for this purpose are disclosed in Bearss et al., U.S. patent application Ser. No. 12/454,062 (US 2009/0298900) (33). One of these compounds is a preferred embodiment and is called Epimerox.

Bacterial and mammalian 2-epimerase enzymes differ due to the presence of an allosteric site on the bacterial enzyme. Such bacteria-specific enzymes can be targeted, e.g. by compounds that inhibit the enzyme, thus disrupting the bacteria without affecting host animals. (33) A unique feature of the bacterial 2-epimerases is their allosteric regulation by a substrate (UDP-GlcNAc), which acts as an activator. Apparently, the allosteric site binds this substrate in order for the enzyme to acquire a conformation that is catalytically competent. This requirement is not found in the mammalian form of the enzyme. (34). One approach for the exclusive targeting of bacteria-specific 2-epimerases is to target the allosteric site, for example by inhibiting its normal binding to the activator substrate. Mammalian 2-epimerases, including human 2-epimerases, which lack the allosteric site, should not be affected, while bacterial 2-epimerases will be disabled or inactivated—resulting in a selective antibacterial agent. Surprisingly, bacteria targeted in this way are less able to develop resistance.

In addition to Epimerox, suitable compounds that target 2-epimerase and evidence reduced potential for resistance can include compounds listed in U.S. patent application Ser. No. 12/454,062, herein incorporated by reference.

### BRIEF DESCRIPTION OF DRAWINGS

FIGS. 1a-e illustrate the interaction of the lysin enzyme PlyG with B. anthracis neutral polysaccharide (NPS), in accordance with an exemplary embodiment of the disclosure.

FIGS. 2a-e illustrate the identification and analysis of 2-epimerase in B. anthracis, in accordance with an exemplary embodiment of the disclosure.

FIG. 3 illustrates a protein sequence alignment of the UDP-GlcNAc 2-epimerases encoded by sps loci of the B. cereus lineage, in accordance with an exemplary embodiment of the disclosure.

FIG. 4 illustrates a protein sequence alignment of the UDP-GlcNAc 2-epimerases encoded by different Gram-positive organisms, in accordance with an exemplary embodiment of the disclosure.

FIG. 5 illustrates a protein sequence alignment of the BA5509 (SEQ ID No. 10) and BA5433 (SEQ ID No. 11) UDP-GlcNAc 2-epimerases encoded by B. anthracis, in accordance with an exemplary embodiment of the disclosure.

FIGS. 6a-b illustrate RT-PCR analysis of BA5509 expression, in accordance with an exemplary embodiment of the disclosure.

FIGS. 7a-c illustrate phenotypic analysis of strains lacking the BA5509- or BA5433-encoded UDP-GlcNAc 2-epimerases of B. anthracis, in accordance with an exemplary embodiment of the disclosure.

FIGS. 8a-b illustrate ultrastructural changes associated with the inhibition or loss of UDP-GlcNAc 2-epimerase activity, in accordance with an exemplary embodiment of the disclosure.

FIGS. 9a-f illustrate antimicrobial activity of Epimerox, in accordance with an exemplary embodiment of the disclosure.

FIGS. 10a-c illustrate the bacterial load in Epimerox treated and untreated mice, in accordance with an exemplary embodiment of the disclosure.

FIG. 11 illustrates Epimerox serial passage experiments, in accordance with an exemplary embodiment of the disclosure.

FIG. 12 illustrates a daptomycin serial passage experiment, in accordance with an exemplary embodiment of the disclosure.

FIG. 13 illustrates an alignment of the 12-amino acid contact points of UDP-GlcNAc in the allosteric site of 2-epimerases in a series of Gram-positive bacteria, in accordance with an exemplary embodiment of the disclosure.

FIG. 14 illustrates the amino acid alignment consensus between the 12-amino acid contact points of UDP-GlcNAc in the allosteric site of 2-epimerases from other bacteria compared to B. anthracis 2-epimerase as shown in FIG. 13, in accordance with an exemplary embodiment of the disclosure.

FIG. 15 illustrates a BLAST analysis of the Gram-positive B. anthracis 2-epimerase with the genome of 2-epimerase for a series of Gram-negative bacteria, in accordance with an exemplary embodiment of the disclosure.

### DETAILED DESCRIPTION OF THE INVENTION

The invention is described below in connection with certain experiments, embodiments and examples, which are representative and serve to illustrate the invention without limiting its scope. Terms used throughout this specification, including particularly technical terms, have their ordinary meanings, in context, within the fields of microbiology and medicine. For clarity, certain terms are specifically defined below.

As used in the specification and the appended claims, the singular forms “a,” “an” and “the” include plural references unless the context clearly dictates otherwise. For example, reference to a component is intended also to include composition comprising a plurality of components. References to a composition containing “a” constituent is intended to include other constituents in addition to the one named.

Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

By “Gram-positive bacteria” it is meant bacteria possessing a peptidoglycan layer comprising tecichoic acid and/or other cell wall associated glycopolymers (CWG), and lacking a cell membrane outside the peptidoglycan layer. By “Gram-negative bacteria” it is meant bacteria possessing a peptidoglycan layer which lacks teichoic acid, and possessing a cell membrane outside the peptidoglycan layer which contains lipopolysaccharides. Gram-positive bacteria can be distinguished from Gram-negative bacteria using a variety of appropriate methods including, but not limited to, growth assays, serological testing, genetic testing and/or microscopy using differential staining techniques. For example, using the “Gram stain” technique, Gram-positive bacteria will retain crystal violet dye, whereas Gram-negative bacteria will not retain crystal violet dye, allowing for color differentiation using microscopy.

When referring to proteins, and more particularly to enzymes, an “active site” denotes any area on an enzyme where a substrate can bind and undergo a chemical reaction. An “allosteric site” denotes any area on an enzyme where an activator or effector can bind and effect the activity of said enzyme.

By “consensus site,” “consensus sequence,” or “consequence motif” it is meant any grouping of nucleotides or amino acids which are at least partially conserved at certain positions within a polynucleotide, or polypeptide, respectively. The grouping of nucleotides or amino acids can represent a consecutive grouping within a single polynucleotide or polypeptide, or a non-consecutive grouping within a single polynucleotide or polypeptide, or a non-consecutive grouping within multiple different polynucleotides or polypeptides.

By “isomerase” it is meant any enzyme that catalyzes the conversion in a biological compound or molecule to a related compound or molecule by changing the stereochemistry at a particular atom within that compound or molecule. By “2-epimerase” it is meant any enzyme which belongs to the family of isomerases which act on carbohydrates and carbohydrate derivatives.

By “treating” or “treatment” is meant any use or administration of any compound or agent for any beneficial or advantageous purpose, including for example to prevent, inhibit, reduce, relieve, or cure any aspect or consequence of any infection or disease condition, including for example a bacterial infection.

By “2-epimerase” or “UDP-GlcNAc 2-epimerase” it is meant a bacterial enzyme which at least catalyses the reversible conversion of UDP-N-acetylglucosamine (UDP-GlcNAc) into UDP-N-acetylmannosamine (UDP-ManNAc). By “relative of 2-epimerase” or “homologue of 2-epimerase” it is meant non-bacterial 2-epimerase enzymes, for example, animal 2-epimerase. By “variant of 2-epimerase” it is meant an isomerase which is structurally distinct from 2-epimerase, but which at least catalyzes the same, or substantially the same, reaction. Preferred variants are those having at least 85%, preferably at least 90%, more preferably at least 95% sequence identity, and most preferably at least 96%, 97%, 98% or 99% sequence identity to a parent wild-type 2-epimerase, such as BA5509 from B. anthracis.

Sequence identity may be determined by any method known in the art, including the use of computer programs for aligning amino acid or nucleic acid sequences, such as BLAST, ALIGN, CLUSTALW, and the like, and unless otherwise stated, using default parameters and taking into account the entire length of each sequence being compared (not just the length of corresponding aligned portions of each sequence).

Any software disclosed herein as being useful in the analysis of data has been used according to procedures typically utilized by those of ordinary skill in the art of the disclosure. Default parameters for the software used herein are suitable. Previous versions of the software as well as later versions of the software are suitable as well as other programs that might be used by one of ordinary skill in the art for analysis of data found within this disclosure.

Many of the proteins described and disclosed herein have been identified by name and ascension number found in GenBank, as maintained by the National Institutes of Health. Sequences from the GenBank are incorporated by reference for the corresponding amino acids disclosed with the ascension number herein.

The invention targets sensitive cell wall proteins of bacteria and enzymes which facilitate essential cell wall functions. Bacteria can be killed, and infectious diseases treated, by interfering with such functions when they are essential to the survival of the microorganism.

An embodiment of the present disclosure includes a family of isomerases known as 2-epimerases. One particular enzyme, 2-epimerase, can be critical in the conversion of a cellular amino sugar, glucosamine, to its related epimer mannosamine. The 2-epimerase enzymes can be found within both animal and bacterial cells. However, bacterial 2-epimerases are not utilized by animal cells, and vice versa.

More specifically, the bacterial 2-epimerases catalyze the reversible conversion of UDP-N-acetylglucosamine (UDP-GlcNAc) to UDP-N-acetylmannosamine (UDP-ManNAc) (35, 36). The latter is an intermediate in the biosynthesis of several bacterial cell surface polysaccharides as well as the enterobacterial common antigen (ECA). The enterococcal common antigen is a surface-associated glycolipid common to all members of the enterobacteriacea family (37). The importance of 2-epimerase in the biosynthesis of polysaccharides in Gram-positive bacteria is highlighted by the presence of two functionally redundant copies of these enzymes in species such as Staphyloccocus aureus and Bacillus anthracis. The bacterial 2-epimerase is related to the bi-functional mammalian UDP-GlcNAc 2-epimerase/ManNAc kinase, a hydrolyzing enzyme that converts UDP-GlcNAc into UDP and ManAc and phosphorylates the latter into ManNAc 6-phosphate (38). The mammalian enzyme catalyzes the rate-limiting step in sialic acid biosynthesis and is a key regulator of cell surface sialylation in humans (39).

A unique feature of the bacterial 2-epimerases is their allosteric regulation by the substrate UDP-GlcNAc, which acts as an activator. In the absence of this activator, virtually no UDP-ManNAc is epimerized in the reverse reaction (34), but when trace amounts of UDP-GlcNAc are added, the reaction proceeds to its normal equilibrium. This suggests that UDP-GlcNAc is required for the enzyme to acquire a conformation in which it is catalytically competent. This requirement is not found in the mammalian form of the enzyme.

The peptidoglycan-linked cell wall glycopolymers (CWGs) of bacteria, including teichoic acids and other secondary cell wall polysaccharides, are gaining interest as targets for antimicrobial drugs (9-11) because of their importance in microbial physiology and virulence. (9, 11-14) According to the invention, CWGs were explored as a target for antimicrobial development using a lysin enzyme called PlyG, which is encoded by a virus that infects the bacteria Bacillus anthracis. More specifically, the γ bacteriophage (or γ phage) of Bacillus anthracis has nucleic acid sequences (viral genes) that express the PlyG lysin at a key point in the life cycle of the phage while it replicates in the bacteria. PlyG cleaves B. anthracis peptidoglycan, a cell wall component of the bacterial that is essential to its structural integrity, in a process proposed (though not proven) to first require PlyG binding to a bacterial neutral polysaccharide (NPS) composed of galactose (Gal), N-acetylglucosamine (GlcNAc) and N-acetylmannosamine (ManNAc). (7, 15)

Importantly, spontaneous resistance to PlyG did not occur in either wild-type B. anthracis (f<5×10⁻⁹ per cell) or in chemically-mutagenized cells with a 1000-fold increase in antibiotic resistance. (7) For this reason, PlyG, can be used to find a CWG in B. anthracis (and its cognate biosynthetic pathway) to serve as a target for antimicrobial development. This is in addition to, and independent of the distinct role of PlyG in the treatment of anthrax, as an antimicrobial agent itself. If spontaneous bacterial resistance to PlyG were not to occur, then chemical inhibitors for the synthesis of its CWG receptor might be less prone to evolving resistance.

Target selection is a critical consideration when developing new antimicrobial agents. It is clearly not sufficient to choose a target based solely on its requirement for viability (i.e., the “classic” method) (24). The need is to identify, first, a target that must be directly or indirectly essential to the virulence or survival of the microorganism, in order for interference with the target to be therapeutically successful, for instance by confronting the target with a ligand, antagonist, inhibitor, drug, etc. Second, the target should be selected, if possible, so that the bacteria has limited alternatives, or no alternatives, to replace the missing function when that target is impaired or disabled. To identify such a target, the more than one billion year co-evolution between bacteria and their phages was taken advantage of by exploiting the lysin-based survival strategy of one B. anthracis-specific phage to identify a cell wall target having little room to vary and evolve resistance.

The identification and selection of the allosteric site of bacterial 2-epimerase provides several advantages in developing antibacterial agents that can capitalize on these criteria. Because the allosteric site of bacterial 2-epimerase does not have a mammalian analog encoded within mammalian 2-epimerase, compound can be developed that would have potentially zero effect on mammalian 2-epimerase. Preferably, an inhibitor of 2-epimerase can be designed and developed that specifically binds to the bacterial 2-epimerase with no binding to the mammalian 2-epimerase. However, compounds that selectively binds to bacterial 2-epimerase in preference to mammalian 2-epimerase are also within the concept of this invention. Therefore, the inhibitors of bacterial 2-epimerase could selectively bind to the bacterial 2-epimerase over the mammaliam 2-epimerase that might encompass suitable ratios of at least about 10:1, at least about 25:1, at least about 50:1, at least about 100:1, t least about 250:1, at least about 500:1, or at least about 1000:1. The inhibitor might also bind in a ratio of at least about 5000:1, 10,000:1, or higher. The inhibitor can bind almost exclusively to bacterial 2-epimerase, and can show almost no binding affinity for mammalian 2-epimerase.

Furthermore, the identification and targeting of the allosteric site of bacterial 2-epimerase allows for inhibition of a bacterial enzyme through a non-active site target, and may play a role in the lack of development of drug resistance. With the targeted allosteric site, an inhibitor of bacterial 2-epimerase can be designed and developed that selectively binds to the allosteric site over the active site. The inhibitor can bind almost exclusively to the allosteric site of the bacterial 2-epimerase, and can have almost no binding affinity for the active site. Alternatively, inhibitors which bind to both sites could still show a preference for the allosteric site. Preferably, the inhibitors would bind specifically to the allosteric site of the bacterial 2-epimerase. However, compounds that selectively prefer the allosteric site over the active site of the 2-epimerase could also be within the concept of this disclosure. For example an inhibitor might selectively bind to the allosteric site over the active site in suitable ratios of at least 2:1, at least about 3:1, at least about 5:1, at least about 10:1, at least about 20:1, at least about 25:1, at least about 33:1, at least about 50:1, at least about 66:1, at least about 75:1, or at least about 100:1. The inhibitor might also selectively bind to the allosteric site over the active site in a suitable ratio of at least about 10:1, at least about 25:1, at least about 50:1, at least about 100:1, at least about 250:1, at least about 500:1, or at least about 1000:1. The inhibitor might also bind in a suitable ratio of at least about 5000:1, 10,000:1, or higher.

Compounds or inhibitors that interact with the allosteric site of the bacterial 2-epimerase can have interactions with the amino acids that create that allosteric site. These interactions are understood by one of ordinary skill to include molecular or atomic level interactions between moieties or atoms of the compound and moieties or atoms of the amino acids. Such interactions can include but are not limited to hydrogen bonding, polar interactions, dipole-dipole interactions, ionic or acid-base interactions, non-polar van der Waals interactions, it electron or aromatic it electron interactions, and so forth. One way of characterizing these interactions is to describe the contact points that the allosteric site exhibits with a compound or inhibitor. Such contact points can be described in terms of the amino acid unit that interacts with the compound or inhibitor. By way of a non-limiting example, UDP-N-acetyl glucosamine can bind and interact with amino acids in the allosteric site of the bacterial 2-epimerase of B. anthracis BA-5509. The UDP-N-acetylglucosamine can demonstrate up to twelve contact points in BA-5509, for contact points at the amino acids Q43, Q46, M47, K67, R69, Q70, T102, E136, R210, E212, and H242. UDP-N-acetylglucosamine can also demonstrate up to twelve contact points with consensus alignment amino acids of the allosteric site of other bacterial 2-epimerase. Similarly, a compound or inhibitor can be designed to interact with some or all of these twelve contact points in an allosteric site of a bacterial 2-epimerase, including at least 3 contact points, at least 4 contact points, at least 5 contact points, at least 6 contact points, at least 7 contact points, at least 8 contact points, at least 9 contact points, at least 10 contact points, at least 11 contact points, or at least 12 contact points. A compound or inhibitor can interact with at least 6 to 8 contact points, at least 8 to 12 contact points, and at least 6 to 12 contact points.

The targeted and structure-based technique described here provided a genus of effective antimicrobial compounds for B. anthracis, including an exemplary compound called Epimerox. These compounds satisfy the Formula I:

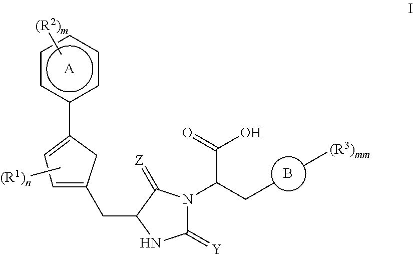

wherein X, Y, and Z each independently is O, S, or NR⁴; A is aryl or hetaryl; or A is halo; B is single-ringed aryl, hetaryl, or hetcyclyl; or B is CH₃; wherein A is halo and B is CH3 cannot occur in same compound; R¹ in each instance independently is C₀₋₄alkyl; R² in each instance independently is C₀₋₄alkyl, C₁₋₄ alkoxy, halo, —CF₂H, —CF₃, —OCF₃, —SCF₃, —SF₅; R³ in each instance independently is C₀₋₄ alkyl; R⁴ in each instance independently is C₀₋₄alkyl, or a single-ringed aryl, hetaryl, or hetcyclyl; n is 0, 1, or 2; and m and mm each independently is 0, 1, 2, 3, 4, or 5; or Formula II

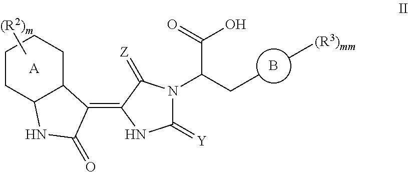

wherein Y, Z each independently is O, S, or NR⁴; A is aryl or hetaryl; B is single-ringed aryl, hetaryl, or hetcyclyl; R² in each instance independently is C₀₋₄ alkyl, C₁₋₄ alkoxy, halo, —CF₂H, —CF₃, —OCF₃, —SCF₃, —SF₅; R³ in each instance independently is C₀₋₄ alkyl; R⁴ in each instance independently is C₀₋₄ alkyl, or a single-ringed aryl, hetaryl, or hetcyclyl; n is 0, 1, or 2; and m and mm each independently is 0, 1, 2, 3, 4, or 5. See, Bearss, U.S. patent application Ser. No. 12/454,062 (US 2009/0298900) (33).

One preferred compound is Formula III, designated Epimerox, and having a chemical name 2-{4-[5-(4-bromophenyl)-thiophen-2-ylmethylene]-5-oxo-2-thioxo-imidazolidin-1-yl}-3-phenyl-propionic acid. (33).

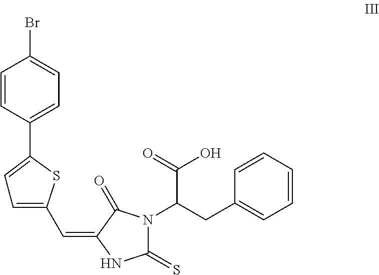 [SMILES: O=C(O)C(Cc1ccccc1)N1C(=O)/C(=C\c2ccc(-c3ccc(Br)cc3)s2)NC1=S]

These compounds, and particularly Epimerox, may be further improved, e.g. with respect to potency, by using a repertoire of lead-optimization methodologies. (25) Bacterial 2-epimerases in general, and perhaps even other enzymes required for the biosynthesis of lysin receptor molecules, could be viable drug targets in other pathogens, e.g. Gram-positive bacteria, for which antibiotic resistance is a problem.

In one embodiment, Gram-negative pathogens may also be targeted by treatment with a compound that can interact with the allosteric site of the bacterial 2-epimerases. As discussed, the Gram-positive bacteria can have a cellular wall comprised of peptidoglycan layer. Gram-negative bacteria also have a peptidoglycan layer associated with the cellular wall, but a lipopolysaccaride layer forms a cellular membrane outside the peptidoglycan wall. Bacterial 2-epimerases that are present in both Gram-positive and Gram-negative bacteria have conserved sequences within their allosteric sites. Thus, a compound that interacts with the allosteric site of a Gram-positive 2-epimerase enzyme may also interact with Gram-negative 2-epimerase enzymes, thereby providing a method for treating bacterial infections across an even broader spectrum of bacteria.

Administering an effective amount comprises delivering an effective amount of at least one inhibitor to a bacterial 2-epimerase at an amount to achieve the desired result, e.g. bacterial inhibition, bacterial cell wall disruption, and so forth. An effective amount is then the amount necessary to invoke the desired effect. The therapeutically effective amount is an amount of the composition that will yield effective results in terms of efficacy of treatment in a given subject. This amount (i.e., dosage) may vary depending upon a number of factors, including, but not limited to, the characteristics of the bacteria, the delivery method, the amount or severity of the bacteria to be inhibited, the physiological condition of the subject (including age, sex, disease type and stage, general physical condition, and responsiveness to a given dosage), the nature of the pharmaceutically acceptable carrier or carriers in the formulation, and the route of administration.

In another aspect of the disclosure, the inhibitors of the invention are a pharmaceutical composition suitable for administration to a mammal, preferably a human. To administer the inhibitors composition to humans or animals, it is preferable to formulate the molecules in a composition comprising one or more pharmaceutically acceptable carriers. The phrase “pharmaceutically or pharmacologically acceptable” refers to molecular entities and compositions that do not produce allergic, or other adverse reactions when administered using routes well-known in the art. “Pharmaceutically acceptable carriers” include any and all clinically useful solvents, dispersion media, coatings, antibacterial and antifungal agents, isotonic and absorption delaying agents and the like.

As used herein the language “pharmaceutically acceptable carrier” includes solvents, dispersion media, coatings, antibacterial and antifungal agents, isotonic and absorption delaying agents, and the like, compatible with pharmaceutical administration. Pharmaceutically acceptable carrier includes any carrier or composition known to one of ordinary skill in the art for administration of the inhibitor, including solid or liquid form, including those adapted for the following: oral administration, for example, drenches (aqueous or non-aqueous solutions or suspensions), tablets (e.g., those targeted for buccal, sublingual, and systemic absorption), capsules, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a spray; sublingually; ocularly; transdermally; pulmonarily; or nasally.

Examples of pharmaceutically acceptable carriers or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used are chosen from, but not limited to, the above or combinations thereof, as appropriate, depending on the dosage form of the present invention.

Reference will now be made in detail to specific aspects of the invention, including compounds, compositions, articles, and methods, examples of which are illustrated in the accompanying Examples and Figures.

### EXAMPLES

### Example 1

### Identification of 2-Epimerase as an Antimicrobial Target

A. Bacterial Strains and Growth Conditions

All strains including S. aureus RN4220 (22), B. anthracis ΔSterne and B. anthracis Sterne (13, 26) were grown in Brain-Heart Infusion broth (BHI; Remel). Strains with the conditional BA5509 mutation (p^{SPAC}-BA5509) were grown overnight in the presence of 1 mM IPTG, washed, diluted 1:100 in BHI with or without IPTG, and grown for the indicated periods of time for analysis. Growth curves were performed in 96-well plates containing 200 μl of culture (with or without IPTG) per well; OD₆₀₀ was recorded every 2 min (40 sec agitation between reads) for 11-20 h at 27° C. in a SpectraMax Plus 96-well plate reader (Molecular Devices).

B. Microscopy.

Phase-contrast and fluorescence microscopy (including use of GFP-PlyG^{BD}) were performed as described using an Eclipse E400 microscope (Nikon) and QCapture Pro version 5.1 software. (26) EM samples were stained in 0.5% uranyl acetate and viewed with a Tecnai Spirit BT Transmission Electron Microscope (FEI). The DeltaVision Image Restoration Microscope (Applied Precision) was used with non-permeabilized cells as described (31); images are deconvolved projections of 3-dimensional data. The following stains were used: PlyG^{BD} coupled to NHS-Rhodamine (Thermo Scientific), 1 μg m1⁻¹; BODIPY FL vancomycin (Invitrogen), 2.5 μg m1⁻¹; DAPI, 2 μg m1⁻¹; and GFP-PlyG^{BD}, 1 μg m1⁻¹. To digest surface proteins, overnight ΔSterne cells were treated for 2 h with chloramphenicol (10 μg m1⁻¹) and proteinase K (100 μg m1⁻¹), washed with PBS, and fixed in 3.75% formalin. Fixed cells were mounted on poly-L-Lysine coated slides and stained with GFP-PlyG^{BD} or anti-Sap antisera and a secondary Alexa Fluor 647-conjugated antibody (Invitrogen, A21245) as described. (31)

C. Preparation and Analysis of Bacterial Cell Wall Carbohydrates.

The isolation and purification of B. anthracis ΔSterne cell walls and subsequent extractions with either SDS or hydrofluoric acid (HF) were performed as described (27) with the exception that bacterial cells were initially disrupted using an EmulsiFlex C5 Homogenizer (Avestin). Glycosyl composition and linkage analyses were performed on the B. anthracis CWG. (15) S. pyogenes CWG was purified as described. (28) For the analysis of PlyG binding to purified wall material, total cell wall, SDS-extracted cell wall, and HF-extracted cell wall stocks were prepared in PBS (5 mg m1⁻¹) and diluted; 70 μl aliquots of the indicated concentrations were then loaded to a dot-blot apparatus (Bio-Rad), transferred to nitrocellulose, and probed with His-tagged PlyG^{BD} (1 mg m1⁻¹). (26) After incubation with Anti-His antibody (Novagen, 70796), binding was visualized using an alkaline phosphatase-conjugated secondary antibody (Sigma, A3563).

D. Lysin Inhibition Assays.

PlyG (70 μl of 7.4 μg m1⁻¹ stock in PBS pH 7.2) and B. anthracis NPS or S. pyogenes CWG (70 μl of indicated concentrations in PBS) were mixed for 30 min at 24° C. in a 96-well plate. Lysin and/or carbohydrate were replaced with PBS alone for controls. After pre-incubation, 70 μl of log phase B. anthracis ΔSterne cells in PBS were added and OD₆₀₀ was monitored every 30 sec (10 sec agitation between reads) for 70 min in a SpectraMax Plus 96-well plate reader. OD₆₀₀ values for PlyG-treated cultures were divided by corresponding values from untreated cultures to evaluate inhibition. For inhibition of PlyG^{BD} binding, 25 μl of PlyG^{BD} (1 μg m1⁻¹) and 25 μl of indicated NPS concentrations were mixed for 30 min at 24° C. before addition of 75 μl of log phase ΔSterne. After 10 min, washed cell pellets were transferred to black 96-well plates to determine relative fluorescence units (RFUs) in a SpectraMax M5 plate reader (Ex=485 nm, Em=538 nm). RFU values for NPS-treated samples were divided by corresponding values for untreated samples to evaluate inhibition.

E. Binding of Lysin to NPS.

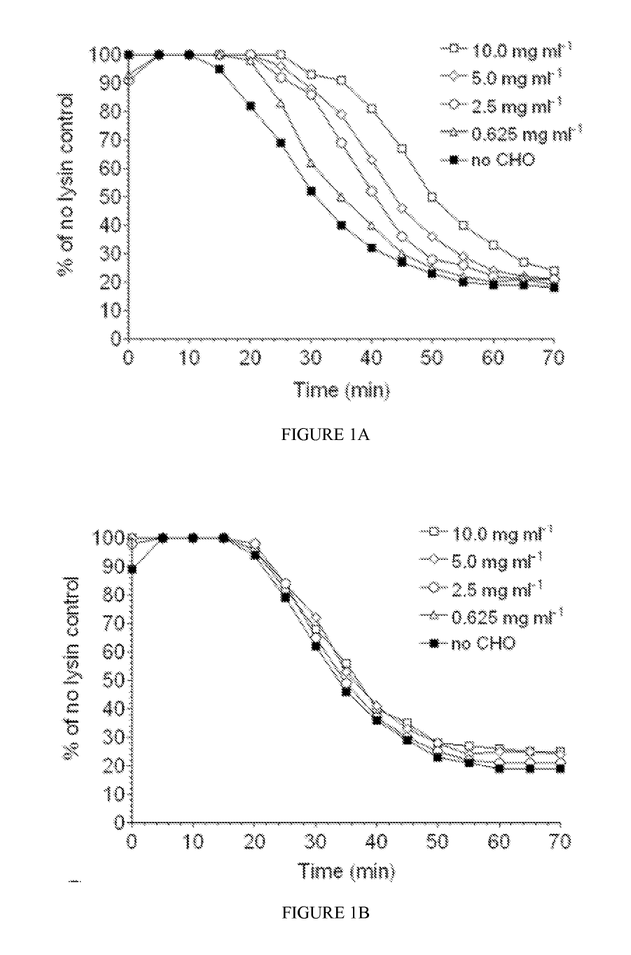

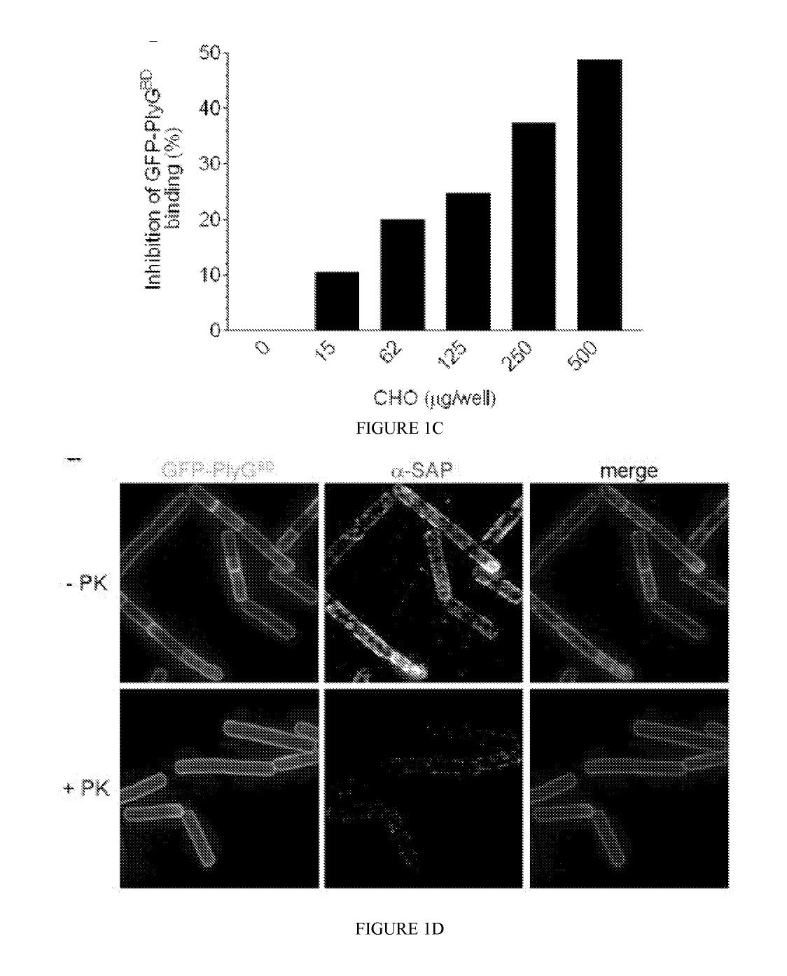

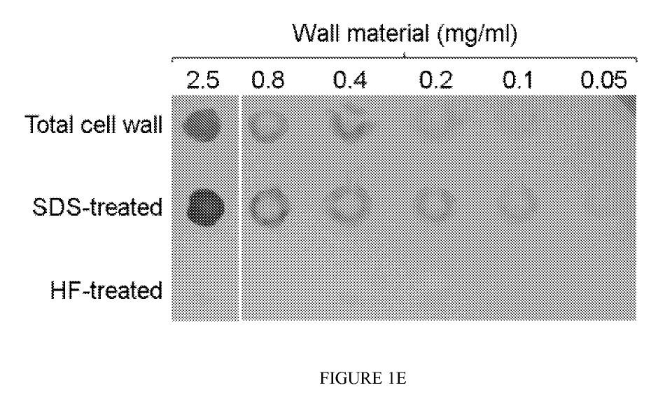

An experiment was conducted to determine whether PlyG binds the B. anthracis NPS. For this, the CWG of B. anthracis strain ΔSterne was purified and subjected to glycosyl composition and linkage analyses to confirm its structure. The extracted material consisted of Gal, GlcNAc, and ManNAc in the 3:2:1 ratio (Table 1) that defines B. anthracis NPS. (15) Methylation analysis also showed glycosyl linkages, including a terminally-linked Gal residue (Table 2), consistent with B. anthracis. (15) Next, pre-incubation of NPS with either PlyG or a GFP-labeled PlyG-binding domain (GFP-PlyG^{BD}) was tested to determine whether ether pre-incubation alters subsequent lytic or cell surface-binding, respectively. See FIGS. 1a-1e for the interaction of PlyG with B. anthracis NPS. (a) Dose-dependent inhibition of PlyG lytic activity after pre-incubation with B. anthracis NPS. (b) PlyG activity after pre-incubation with increasing amounts of the CWG from Streptococcus pyogenes. (c) Dose-dependent inhibition of PlyG^{BD} surface-binding after pre-incubation with B. anthracis NPS (d) Deltavision images of surface-labeled B. anthracis with or without proteinase K treatment (+/−PK). NPS (green) was labeled with GFP-PlyG^{BD}, and the S-layer Sap protein (red) was labeled with specific antibodies and an Alexa Fluor 647-conjugated secondary antibody. (e) Dot-blot analysis of PlyG^{BD} binding to total cell wall material and both SDS-treated and HF-treated walls. Dose-dependent responses were observed in both cases, with increasing NPS levels blocking PlyG-directed lysis and binding (FIGS. 1a and c). Pre-incubation of PlyG with the CWG of Streptococcus pyogenes (a structure unrelated to B. anthracis NPS), however, had no effect on lytic activity (FIG. 1b). As proof that PlyG does not bind a protein receptor, it was also found that GFP-PlyG^{BD} labels proteinase K-treated bacteria lacking most surface proteins (including the S-layer protein, Sap) (FIG. 1d). Additionally, His-tagged PlyG^{BD} binds in a dose-dependent manner to purified B. anthracis cell wall material and SDS-treated walls (lacking most surface proteins), but not to walls extracted with hydrofluoric acid to remove CWGs (FIG. 1e). Together, these findings suggest that NPS is the PlyG cell wall receptor.

TABLE 1
Sugar composition of cell walls of strains used in this study.

| | Sugar composition (%)* | | | | | |
|---|---|---|---|---|---|---|
| Strain | Man | Fuc | Glc | Gal | ManNAc | GlcNAc |
| B. anthracis ΔSterne | ND | ND | ND | 49.9 | 16.5 | 33.6 |

*Values are expressed as mole percent of total carbohydrate. The sample was 99% carbohydrate. ND, none detected (i.e., <0.5%). Abbreviations are as follows: Man, mannose; Fuc, fucose; Glc, glucose; Gal, galactose; ManNAc, N-acetyl mannosamine; GlcNAc, N-acetyl glucosamine. Analysis was performed by combined gas chromatography/mass spectrometry (GC/MS) of the per-O-trimethylsilyl derivatives of the monosaccharide methyl glycosidase produced from the samples by acidic methanolysis.

TABLE 2
Glycosyl linkage analysis.

| | % Present* |
|---|---|
| | B. anthracis |
| Glycosyl residue | ΔSterne |
| Terminally linked galactopyranosyl residue (T-Gal) | 78.4 |
| 3-linked galactopyranosyl residue (3-Gal) | ND |
| 6-linked galactopyranosyl residue (6-Gal) | 1.3 |
| Terminally linked N-acetyl glucosamine residue | ND |
| (T-GlcNAc) | |
| 4-linked N-acetyl glucosamine residue (4-GlcNAc) | 4.0 |
| 4-linked N-acetyl mannosamine residue (4-ManNAc) | 2.7 |
| 6-linked N-acetyl glucosamine residue (6-GlcNAc) | ND |
| 3,4-linked N-acetyl mannosamine residue (3,4-ManNAc) | ND |
| 3,4-linked N-acetyl glucosamine residue (3,4-GlcNAc) | 10.3 |
| 4,6-linked N-acetyl glucosamine residue (4,6-GlcNAc) | 2.4 |
| 4,6-linked N-acetyl mannosamine residue (4,6-ManNAc) | ND |
| 3,4,6-linked N-acetyl mannosamine residue | 0.9 |
| (3,4,6-ManNAc) | |

*For glycosyl linkage analysis, the sample was permethylated, depolymerized, reduced, and acetylated; the resultant partially methylated alditol acetates (PMAAs) were analyzed by GC-MS.
ND, none detected.

F. Contrasting the Biosynthetic Pathways of PlyG-Sensitive and PlyG-Insensitive for B. Anthracis NPS.

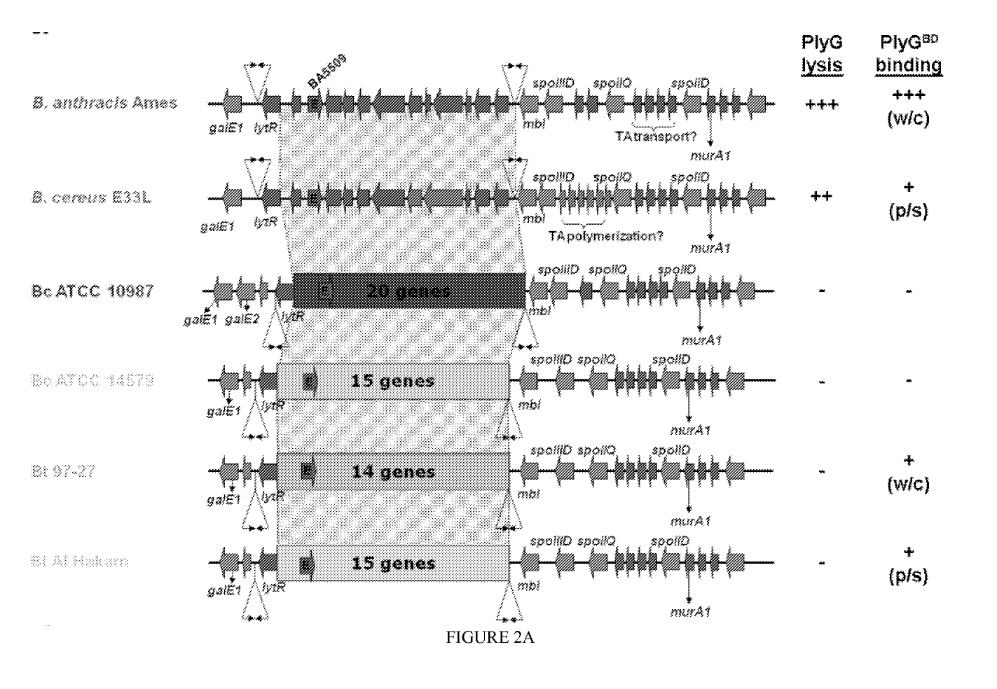

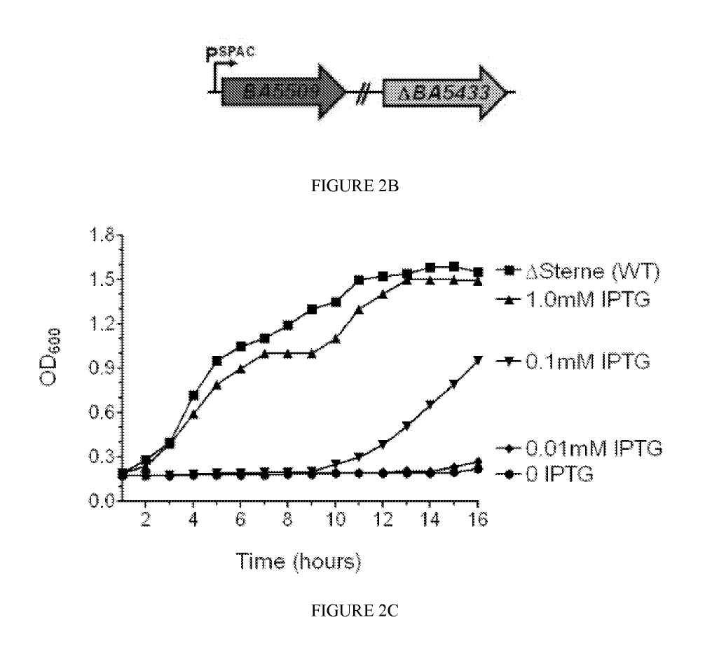

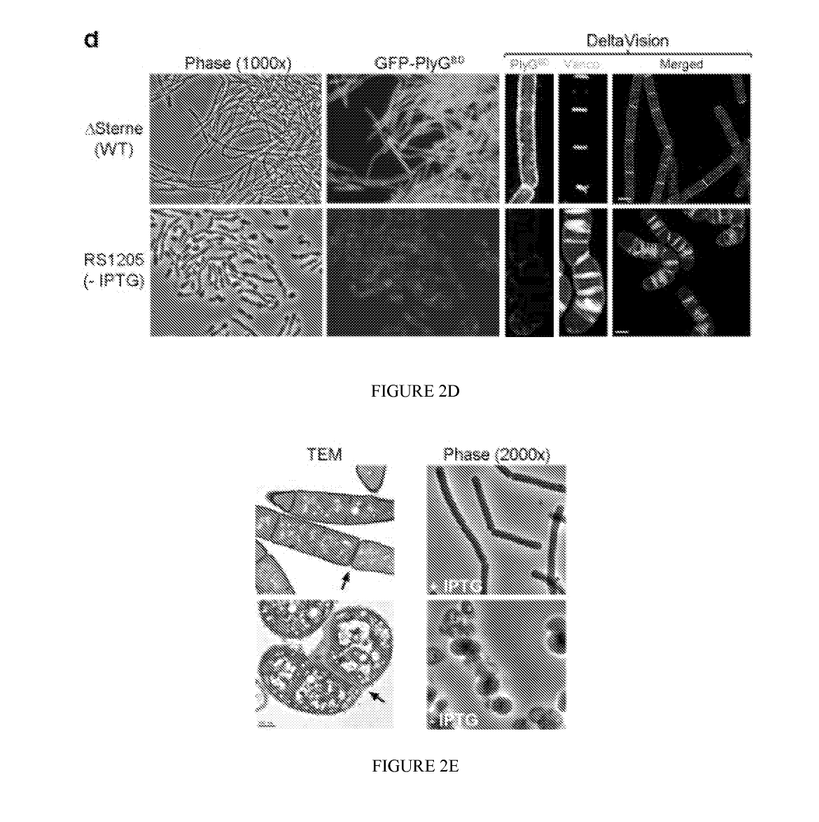

A direct genomic comparison of the PlyG-sensitive B. anthracis Ames strain and the genetically related but PlyG-resistant strain B. cereus 10987 (7, 16), revealed an Ames-specific gene cluster annotated as a CWG biosynthethic pathway. See FIGS. 2a-2e, for identification and analysis of 2-epimerase in B. anthracis. (a) sps loci of the B. cereus lineage. Islands of variable sps genes are connected by gray regions and denoted by different colors. Conserved flanking sequences are shown. Red shaded loci (not in Ames) are cell wall-biosynthetic genes similar to that encoded by Ames. Inverted arrows are repeat elements. Susceptibility to PlyG lysis and GFP-PlyG^{BD} surface binding are shown. Abbreviations: w/c, whole-cell binding; p/s, polar/septal binding. (b) Genetic representation of 2-epimerase double mutant, RS1205. (c) Growth of RS1205 (with indicated IPTG concentrations) compared to the parental wild-type strain ΔSterne. Mean averages are shown (n=3) with standard deviations. (d) Morphological analysis of RS1205 after five hours of growth without IPTG. Phase contrast images and corresponding fluorescence fields are shown for GFP-PlyG^{BD}-labeled RS1205 (5 second exposure) and B. anthracis ΔSterne (30 second exposure). For Deltavision images, NPS (red) was labeled with rhodamine-PlyG^{BD}, division septa (green) were labeled with vancomycin BODIPY FL, and DNA (blue) was labeled with DAPI. TEM images are shown with scale bars (500 nm) and arrows denote some division septa. (e) Phase contrast microscopic images of RS1205 grown for 12 hours with and without IPTG (5 μM). Corresponding to the ˜16 kb BA5508-BA5519 locus in B. anthracis, the size and gene content of this region (defined as sps, for surface polysaccharide synthesis) was remarkably variable over a wide range of highly related B. cereus group organisms (FIG. 2a; Tables 3 and 4). All sps loci are encoded on genetic “islands” with G+C contents distinct from their background genomes, and are flanked by nearly identical DNA sequences extending at least 5-10 kb (Table 4 and 5). Variation in sps content likely explains why CWGs with related, yet distinct glycosyl compositions are found throughout the B. cereus group. (17) Interestingly, B. cereus strain E33L, with sps locus 61% identical to that of B. anthracis, is also sensitive to PlyG (FIG. 2a). These findings support the idea that the B. anthracis sps locus specifies the production of NPS.

TABLE 3
The position and size of sps loci in strains from this study.

| | Accession | sps size | | |
|---|---|---|---|---|
| Organism | number | (bp) | sps start* | sps end |
| B. anthracis Ames | AE016879 | 16,659 | 4,995,104 | 5,011,763 |
| B. cereus E33L | CP000001 | 16,115 | 5,056,954 | 5,073,069 |
| B. cereus 10987 | AE017194 | 19,866 | 4,970,969 | 4,990,835 |
| B. cereus 14579 | AE016877 | 16,217 | 5,174,133 | 5,190,530 |
| B. thuringiensis 97-27 | AE017355 | 14,713 | 5,001,698 | 5,016,411 |
| B. thuringiensis Al Hakam | CP000485 | 16,021 | 5,024,784 | 5,040,805 |

*Genomic positions (according to the indicated GenBank sequences) of the 5′ end of the first sps gene and the 3′ end of the last sps gene are reported. The sps locus consists of all loci between lytR and mre. The first sps gene for each strain is: BA5508, Ames; BCZK4963, E33L; BCE_5384, ATCC 10987; BC5266, ATCC 14579; BT9727_4948, 97-27, and BALH_4769, Al Hakam. The last sps gene for each strain is: BA5519, Ames; BCZK4979, E33L; BCE_5403, ATCC 10987; BC5280, ATCC 14579; BT9727_4961, 97-27, and BALH_4784, Al Hakam.

TABLE 4
Sequence comparisons of sps loci (and flanking regions).

| | % identity to B. anthracis Ames loci | | | PlyG |
|---|---|---|---|---|
| | 10 kb | sps | 10 kb | sensi- |
| Strain | left* | locus | right** | tivity |
| B. cereus E33L | 95 | 61 | 97 | + |
| B. cereus 10987 | 84 | 7 | 90 | − |
| B. cereus 14579 | 90 | 7 | 91 | − |
| B. thuringiensis 97-27 | 96 | 7 | 97 | − |
| B. thuringiensis Al Hakam | 97*** | 7 | 98 | − |

*The left end regions of homology are defined according to positions in GenBank sequences as follows: 4,981,947-4,991,947 in Ames; 5,043,808-5,053,808 in E33L; 4,956,643- 4,966,643 in ATCC 10987; 5,160,984-5,170,984 in ATCC 14579; 4,988,569-4,998,569 in 97-27; and 5,006,504-5,016,504 in Al Hakam. The starting point adjacent to sps was chosen, in each case, as the locus immediately downstream of galE1.
**In right end regions of homology are defined as follows: 5,014,709-5,024,709 in Ames; 5,084,089-5,094,089 in E33L; 4,992,880-5,002,880 in ATCC 10987; 5,194,047-5,204,047 in ATCC 14579; 5,018,985-5,028,985 in 97-27; 5,043,242-5,053,242 in Al Hakam. The starting point adjacent to sps was chosen, in each case, as the 3′ end of spollQ.
***The left-end region of homology between Ames and Al Hakam only extends 5 kb. The value here denotes the % identity over this 5 kb region.

TABLE 5
The G + C content of sps loci (and it flanking regions).

| | % G + C | | | |
|---|---|---|---|---|
| | Total | 10 kb | sps | 10 kb |
| Strain | genome* | left** | locus | right*** |
| B. anthracis Ames | 35.4 | 37.47 | 31.95 | 37.84 |
| B. cereus E33L | 35.4 | 37.51 | 33.03 | 37.96 |
| B. cereus 10987 | 35.6 | 38.12 | 32.18 | 38.33 |
| B. cereus 14579 | 35.3 | 37.28 | 31.93 | 37.39 |
| B. thuringiensis 97-27 | 35.4 | 37.34 | 31.85 | 37.78 |
| B. thuringiensis Al Hakam | 35.4 | 38.45 | 32.30 | 37.89 |

*The total chromosomal G + C content of each strain listed here was taken from the website http://insilico.ehu.es/oligoweb/index2.php?m=all.
**The chromosomal positions of left flanking regions were identical to that listed in Table 3. The value for the left end region of Al Hakam represents only 5 kb of flanking sequence.
***The chromosomal positions of right flanking regions were identical to that listed in Table 3.

G. Identification of an Antimicrobial Target.

One protein, encoding a putative non-hydrolyzing UDP-N-acetylglucosamine

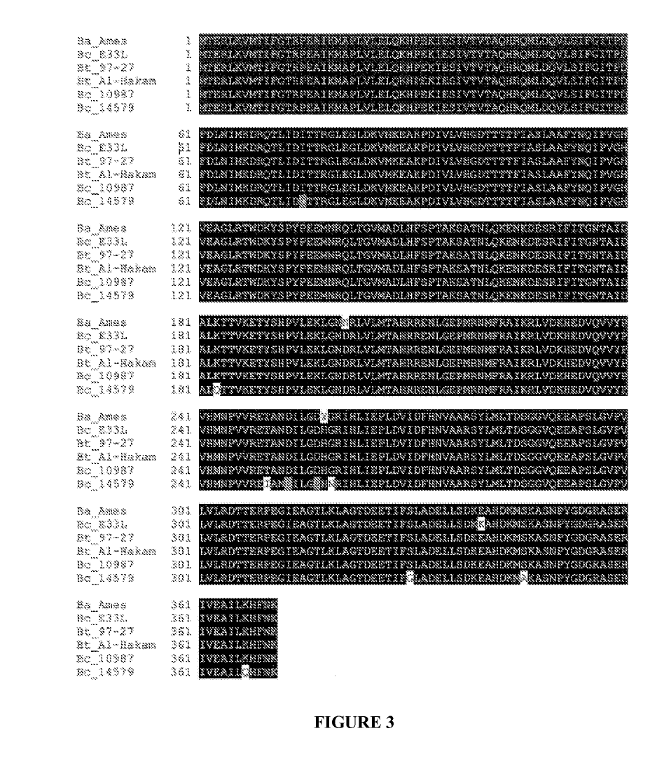

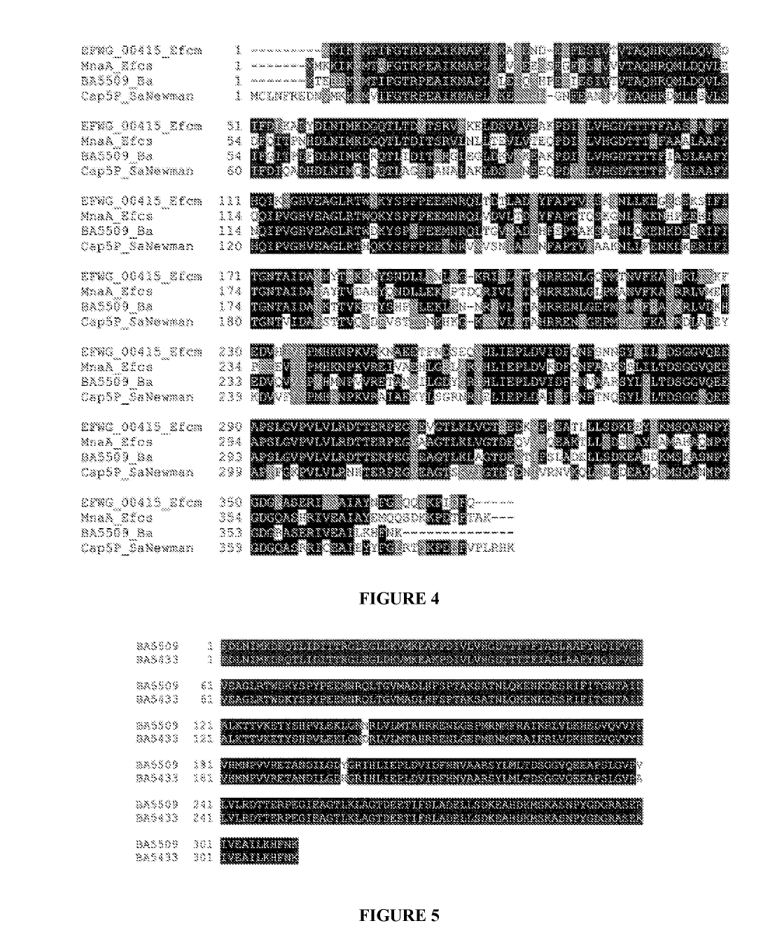

2-epimerase (or 2-epimerase), was conserved among the otherwise distinct sps loci in the B. cereus group (FIG. 2a). The 2-epimerases are >98% identical within the B. cereus group and >60% identical over a range of Gram-positive organisms See FIG. 3, for the protein sequence alignment of the UDP-GlcNAc 2-epimerases encoded by sps loci of the B. cereus lineage. Alignments were obtained using ClustalW. Shading was generated by Boxshade. Black indicates 100% identical residues and gray indicates conserved amino acid changes. Proteins included are as follows: BA5509 in B. anthracis Ames (SEQ ID No. 1), MnaA in B. cereus E33L (SEQ ID No. 2), BCE 5307 in B. cereus ATCC 10987 (SEQ ID No. 3), BC5201 in B. cereus ATCC 14579 (SEQ ID No. 4), BT9727 4878 in B. thuringiensis 97-27 (SEQ ID No. 5), and BALH_{—}4693 in B. cereus Al Hakam (SEQ ID No. 6). FIG. 4 for protein sequence alignment of the UDP-GlcNAc 2-epimerases encoded by different Gram-positive organisms. Alignments were obtained using ClustalW. Shading was generated by Boxshade. Black indicates 100% identical residues and gray indicates conserved amino acid changes. Proteins included are as follows: BA5509 in B. anthracis strain Ames (SEQ ID No. 1), EFWG_{—}00415 in Enterococcus faecium strain Conn15 (SEQ ID No. 7), MnaA (or HMPREF0348_{—}1199) in E. faecalis strain TX0104 (SEQ ID No. 8), and Cap5P (or NWMN_{—}0110) in S. aureus strain Newman (SEQ ID No. 9). Bacterial 2-epimerases convert UDP-GlcNAc into UDP-ManNAc prior to the polymerization of CWG subunits; epimerization is an early reaction in CWG biosynthesis and can be important or essential for growth. (18, 19) Considering the importance of 2-epimerases for bacterial viability, the broad distribution of such enzymes, and the presence of ManNAc in the B. anthracis lysin-inhibiting NPS, the 2-epimerse encoded by BA5509 was chosen for further characterization.

To investigate BA5509 as an antimicrobial target, the importance of 2-epimerase to the viability of B. anthracis was evaluated. A caveat of mutant construction, however, concerned the fact that B. anthracis encodes a second 2-epimerase, BA5433, which is 99% identical to BA5509. FIG. 5 illustrates a protein sequence alignment of the BA5509 (SEQ ID No. 10) and BA5433 (SEQ ID No. 11) UDP-GlcNAc 2-epimerases encoded by B. anthracis. Alignments were obtained using ClustalW. Shading was generated by Boxshade. Black indicates 100% identical or conserved residues. The BA5509 promoter was replaced with the IPTG-inducible P^{SPAc} promoter as described. (29) Briefly, the first 471 bases of BA5509 and its preceding ribosome binding site were PCR amplified with BA5509 mutagenesis primers (Table 6). Primer-encoded attB1 and attB2 recombinase recognition sites permitted cloning into the Gateway vector pDONRtet (Invitrogen) and transfer into pNFd13. Transformation of ΔSterne and integration into BA5509 was performed in the presence of 5 mM IPTG. Disruption of BA5433 was performed as described (30), using a 190 bp internal PCR fragment amplified with BA5433 mutagenesis primers and cloned into the Kpn1 site of plasmid pASD4. RT-PCR analysis of RS1205 was performed as described (26), using the primers in Table 6. Quantitative PCR (qRT-PCR) analysis was performed as described³¹ using primers in Table 6 and probes for BA5509 (5′-CCGTCGTGAAAACTT-3′) (SEQ ID NO. 37) and the housekeeping gene rpoB (5′-CTGCCGCTAAAATTT-5′) (SEQ ID NO.38); rpoB served as the internal control for gene expression.

TABLE 6
Primers used in this study.

| Gene | Upstream (5′-3′) | Downstream (5′-3′) |
|---|---|---|
| BA5509 (RT-PCR) | taatggcggaccttcatttc | caagaaccggtacaccaagtga |
| | (SEQ ID NO. 39) | (SEQ ID NO. 40) |
| BA5510 (RT-PCR) | gttggaattgtaggtttaaatggttctg | ggaacagtggatattaaaggttcagc |
| | (SEQ ID NO. 41) | (SEQ ID NO. 42) |
| BA5511 (RT-PCR) | ccagtacatggcgttccttactt | agagctccgcgatatacttctac |
| | (SEQ ID NO. 43) | (SEQ ID NO. 44) |
| BA5509 | ggggacaagtttgtacaaaaaagcaggct- | ggggaccactttgtacaagaaagctgggt- |
| (mutagenesis)* | catgtataataatacagtaacaatactaccaga | gaaggtccgccattacgcctg |
| | (SEQ ID NO. 45) | (SEQ ID NO. 46) |
| BA5433 | Gtaggtaccggcacctcttgtattagagttg | Gtaggtacccaacacgaggtttagaaggtttg |
| (mutagenesis)** | (SEQ ID NO. 47) | (SEQ ID NO. 48) |
| BA5509 (qRT- | cgtactagagaaacttggaaataatcgtctt | gcacggaacatattacgcattgg |
| PCR) | (SEQ ID NO. 49) | (SEQ ID NO. 50) |
| rpoB (qRT-PCR) | agctgaaacattagtagatccagaaactg | aatgcgatcaagtgtacgacgat |
| | (SEQ ID NO. 51) | (SEQ ID NO. 52) |

*Bolded sequences represent attB1 and attB2 sites.
**Bolded sequences represent Kpnl sites.

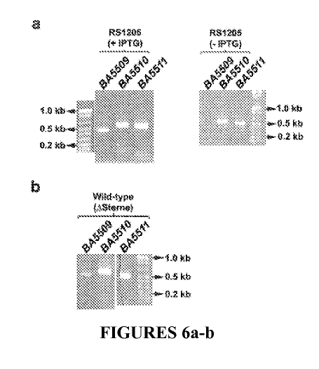

The potential for functional redundancy required construction of a BA5509-BA5433 double mutant (strain RS1205) (FIG. 2b), in addition to single mutants. A conditional 2-epimerase mutant was first generated by placing the wild-type, monocistronic BA5509 locus under IPTG-inducible SPAC promoter control. BA5433 was then inactivated, in both wild-type and BA5509 mutant backgrounds, by chromosomal integration of a recombinant plasmid. For the RS1205 double mutant, RT-PCR confirmed the IPTG-dependence for BA5509 expression and the fact that the BA5509 mutation did not affect expression of downstream, divergently transcribed sps genes. FIGS. 6a and 6b show RT-PCR analysis of BA5509 expression. RNA was prepared after 5 hours of growth in BHI medium with or without 5 mM IPTG. cDNA was then generated and analyzed by PCR with primers specific for the indicated loci. (a) Expression of BA5509 (and the downstream loci BA5510 and BA5511) in the 2-epimerase double-mutant strain RS1205. (b) Gene expression in the wild-type B. anthracis strain ΔSterne. DNA size standards are shown.

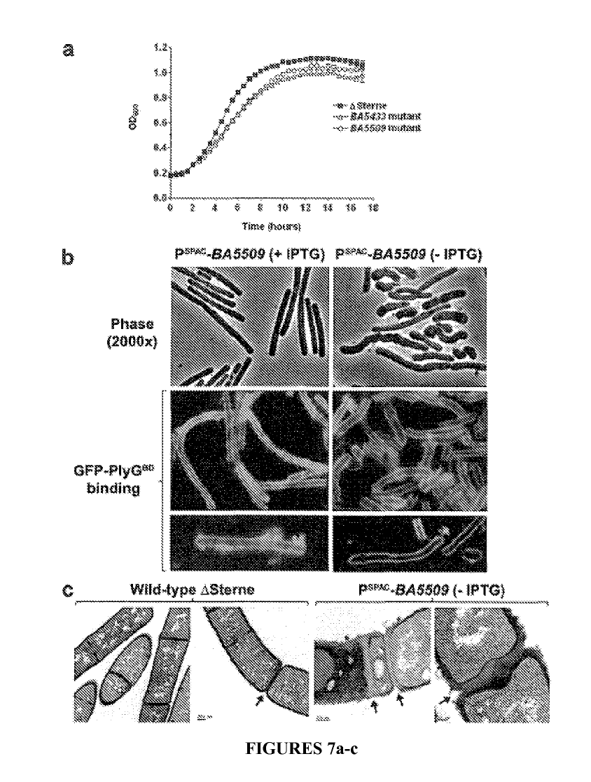

FIGS. 7a-c show phenotypic analysis of strains lacking the BA5509- or BA5433-encoded UDP-GlcNAc 2-epimerases of B. anthracis. The BA5509 mutant, also referred to as P^{SPAC}-BA5509, was grown with 5 mM IPTG unless otherwise indicated.

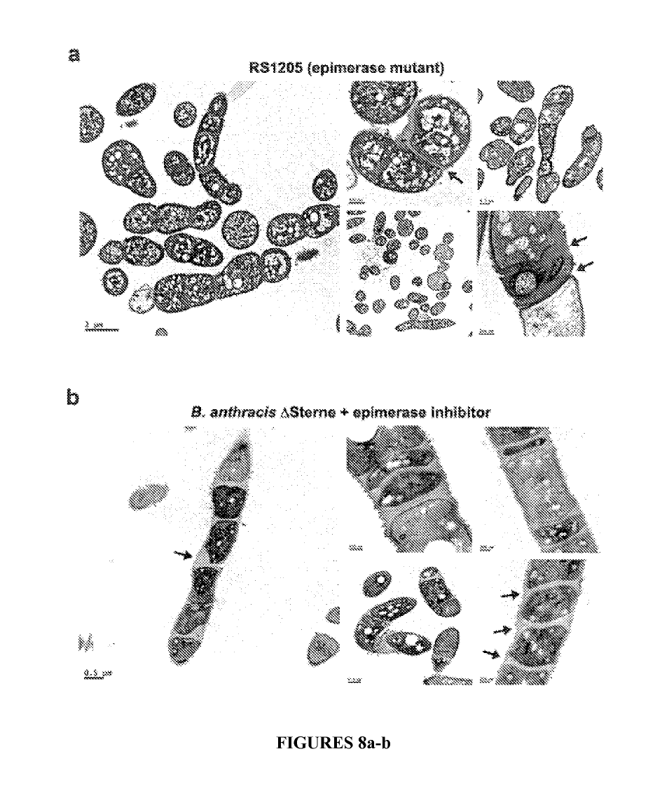

FIG. 7A shows Growth curve in BHI medium. FIG. 7B shows the phase contrast and fluoresence microscopic analysis of strains grown for 10 hours. FIG. 7C shows the transmission electron micrographs of strains grown for 10 hours in BHI. Scale bars are 200 nm and arrows denote some division septa. The loss of either BA5509 or BA5433 alone had a slight impact on B. anthracis growth (FIG. 7a). While the BA5509 single mutant did have bulging cell walls and septation (partitioning) at inappropriate sites, GFP-PlyG^{BD} binding to surface NPS was largely unaffected (FIG. 7b and c). The RS1205 double mutant, on the other hand, had substantial growth and morphological defects. In media supplemented with decreasing IPTG concentrations, the growth of RS1205 was arrested at 0.01 mM IPTG (FIG. 2c). Microscopic examination of RS1205 revealed a progression from typical rod-shaped forms into coccoid cell-aggregates after 5 and 12 hours without IPTG (FIG. 2d and e, FIG. 8a), in a process marked by aberrant septation and the near absence of PlyG^{BD}-labeling of cell-surface NPS. FIGS. 8a and 8b show ultrastructural changes associated with the inhibition or loss of UDP-GlcNAc 2-epimerase activity. Scale bars are shown and arrows denote some division septa. (a) The B. anthracis ΔSterne epimerase mutant derivative RS1205 (P^{SPAC}-BA5509/BA5433::pASD4) grown for 12 hours in the absence of IPTG. (b) B. anthracis ΔSterne treated with Epimerox (5 μM) for 5 hours at 30° C. with aeration. Conversion into unstable coccal forms is a hallmark of mutants deficient in CWG synthesis. (11, 18, 19). These results imply that 2-epimerase is required for NPS synthesis and is important, if not essential, for B. anthracis viability.

### Example 2

### Demonstration of Inhibitor Identification for a Microbial Target

A. Virtual Screening Assay.

Stage 1 hit-finding was initiated using the allosteric site in the BA5509 2-epimerase crystal structure as a model for docking a virtual library of ˜2 million small molecules and generating a subset of hits, based on calculated binding energies. The performance and pharmacologic activity of stage 1 hits were evaluated using physicochemical and ADMET (Absorption, Distribution, Metabolism, Excretion, and Toxicity) prediction algorithms. One-hundred compounds from the virtual screening set were then screened in both a biochemical assay²⁰, and the B. anthracis growth inhibition assay. Numerous compounds were ultimately identified based on the ability to inhibit B. anthracis growth by over 50% (compared with untreated controls) at a concentration of 30 μM. These lead candidates served as starting points for optimization. Based on CLIMB® guided design, 62 compounds were synthesized and tested for B. anthracis growth inhibition. Epimerox, the most potent inhibitor, was chosen for further pharmacological evaluation.

B. Growth Inhibition Assays.

Previously, the crystal structure of BA5509 was solved, and a novel regulatory mechanism requiring direct interaction between identical substrate molecules (UDP-GlcNAc in this case) in both the active and allosteric sites was identified. (20) Having validated BA5509 as a target for antimicrobial development, the BA5509 structure (particularly the allosteric and active site residues conserved among bacterial 2-epimerases such as those found in BA5509 and BA5433, but not present in the human equivalent enzymes), were used as the basis for identifying inhibitory molecules. The BA5509 active and allosteric sites were first used in a docking model for a virtual library of ˜2,000,000 small molecules.

A subset of initial compounds was identified based on calculated binding energies and predictive models for suitable drug candidates, and synthesized for testing in a B. anthracis growth inhibition assay. Thirty compounds, active at 30 μM, were chosen for optimization, eventually yielding 62 additional compounds for testing. Assay of the compounds were conducted using standard techniques, such as disclosed in reference 10. Table 7 discloses the initial 30 compounds and assay values. Table 8 discloses the additional 62 compounds and associated assay values.

C. Growth Inhibition by Epimerox.

The most potent inhibitor in these experiments, called Epimerox, is an oxo-imidizolyl compound. One millimolar Epimerox stock solutions in 5 mM DMSO were diluted into assays at indicated concentrations. Final DMSO concentrations were always 3%. Wells of a 96-well plate contained 6 μl of inhibitor (or 6 μl of DMSO as a control), 94 μl of BHI, and 100 μl of log phase cells (OD₆₀₀ 0.2) in BHI. OD₆₀₀ was recorded in a SpectraMax Plus 96-well plate reader at 28° C. with agitation every 2 min. Growth inhibition was calculated as follows:

$$\frac{\begin{matrix}100 ( 1 - [ ( {OD}_{600} at endpoint of inhibitor culture - \\ {OD}_{600} of media background )\end{matrix}}{\begin{matrix}( {OD}_{600} at endpoint of DMSO control culture - \\ {OD}_{600} of media background ) ] )\end{matrix}}$$

Endpoint was defined as the entry point into stationary phase. Assays were performed in triplicate.

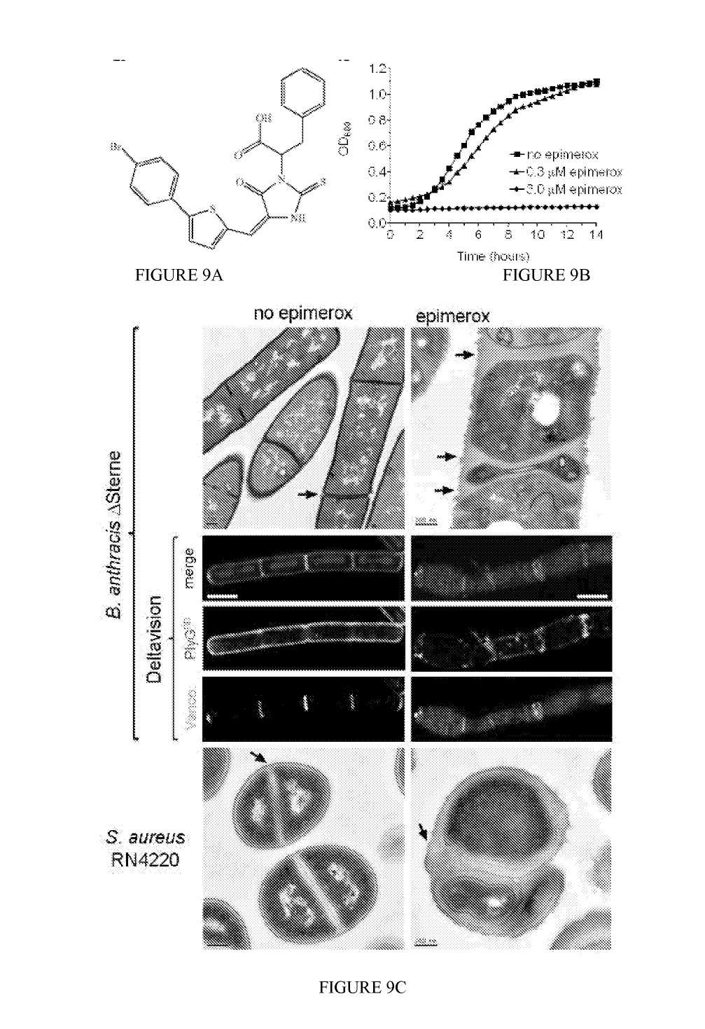

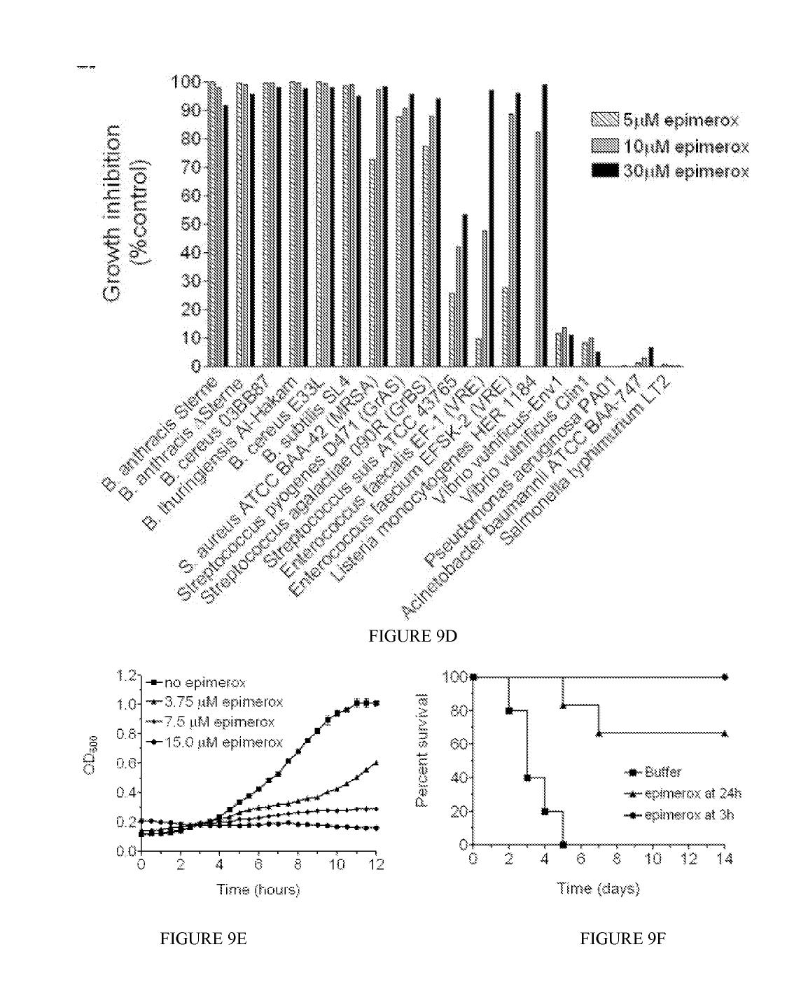

Epimerox demonstrated a minimum inhibitory concentration (MIC) of 4.0 μg m1⁻¹ (7.6 μM) against both B. anthracis Sterne and ΔSterne strains (Table 9). Considering the well-described genetic homogeneity of all B. anthracis isolates (21) and the 100% identity of BA5433 and BA5509 protein sequences from over 30 distinct members of the B. cereus lineage of organisms, it is likely that all isolates would be susceptible to Epimerox. FIGS. 9a-f demonstrate the antimicrobial activity of Epimerox: (a) Chemical structure of Epimerox. (b) Growth curves of B. anthracis ΔSterne in BHI medium with and without Epimerox. (c) Morphologies of B. anthracis and S. aureus after 5 hours of exposure to Epimerox (5 μM and 14 μm, respectively). For Deltavision images, NPS (red) was labeled with rhodamine-PlyG^{BD}, division septa (green) were labeled with vancomycin BODIPY FL, and DNA (blue) was labeled with DAPI. For TEM images, arrows indicate some division septa. Scale bars are shown. (d) Growth inhibition assays for Gram-positive and -negative organisms. Cultures were grown in BHI medium with and without indicated Epimerox concentrations for 11 hours at 28° C. (e) Growth curves of S. aureus strain RN4220 in BHI medium with and without Epimerox. (f) Survival plot of C57BL/6 mice after i.p. infection with 5×10⁵ CFUs of B. anthracis Sterne, and i.p. treatment with buffer starting at 3 hours post-infection (and continuing every 6 hours for 7 days), or Epimerox (13 mg/kg) starting at 3 hours or 24 hours post-infection (and continuing every 6 hours for 7 days).

When added to B. anthracis cultures, Epimerox effectively blocked growth for up to 14 hours at concentrations above 3 μM (FIG. 9b). As with the 2-epimerase double mutant RS1205, microscopic analysis revealed a conversion from rod-shaped forms into swollen and rounded cell types after inoculation into media containing 5 μM Epimerox (FIGS. 8b and 9c). Growth inhibition was associated with a dramatic reduction in PlyG^{BD} surface-binding and the formation of aberrant division septa (FIG. 9c). These findings are consistent with Epimerox acting as a bacteriostatic agent through the inhibition of 2-epimerase activity and NPS biosynthesis in B. anthracis and are consistent with the phenotype seen in cells lacking both 2-epimerase genes.

TABLE 7

| | | B. Anthracis | | | S. MRSA | | |
|---|---|---|---|---|---|---|---|
| Cmpd | Structure | 30 μM | 10 μM | 3 μM | 30 μM | 10 μM | 3 μM |
| 1 | 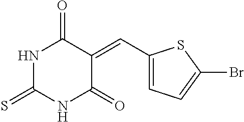 [SMILES: O=C1NC(=S)NC(=O)C1=Cc1ccc(Br)s1] | 8.74 | −12.36 | | 22.06 | 6.62 | |
| 2 | 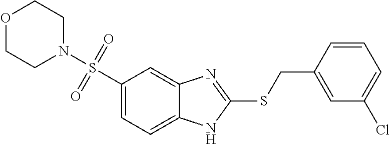 [SMILES: O=S(=O)(c1ccc2[nH]c(SCc3cccc(Cl)c3)nc2c1)N1CCOCC1] | 37.26 | 11.98 | 0 | 46.21 | −24.79 | |
| 3 | 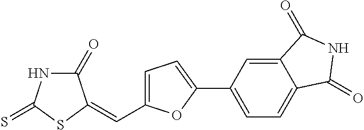 [SMILES: O=C1NC(=S)S/C1=C/c1ccc(-c2ccc3c(c2)C(=O)NC3=O)o1] | 87.45 | 13.69 | | 61.18 | −0.59 | |
| 4 | 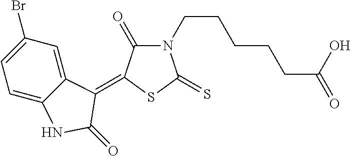 [SMILES: O=C(O)CCCCCN1C(=O)/C(=C2/C(=O)Nc3ccc(Br)cc32)SC1=S] | 91.25 | 52.28 | | 69.41 | 41.62 | |
| 5 | 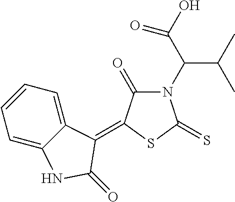 [SMILES: CC(C)C(C(=O)O)N1C(=O)/C(=C2/C(=O)Nc3ccccc32)SC1=S] | 11.21 | −1.33 | | 40.59 | 15.88 | |
| 6 | 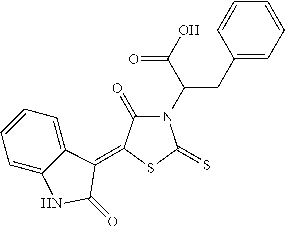 [SMILES: O=C1Nc2ccccc2/C1=C1/SC(=S)N(C(Cc2ccccc2)C(=O)O)C1=O] | 92.58 | 19.58 | | 47.79 | 45.74 | |
| 7 | 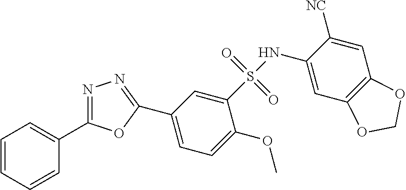 [SMILES: COc1ccc(-c2nnc(-c3ccccc3)o2)cc1S(=O)(=O)Nc1cc2c(cc1C#N)OCO2] | 74.9 | 19.58 | | -1.62 | −7.79 | |
| 8 | 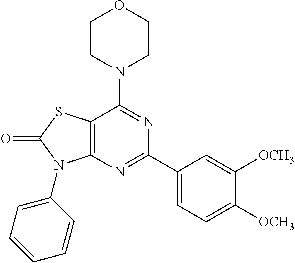 [SMILES: COc1ccc(-c2nc(N3CCOCC3)c3sc(=O)n(-c4ccccc4)c3n2)cc1OC] | 35.55 | 5.32 | | 1.47 | −8.82 | |
| 9 | 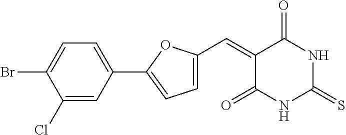 [SMILES: O=C1NC(=S)NC(=O)C1=Cc1ccc(-c2ccc(Br)c(Cl)c2)o1] | 88.4 | 27.96 | | −4.22 | 18.97 | |
| 10 | 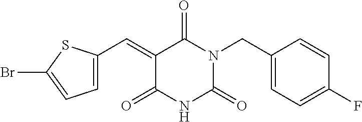 [SMILES: O=C1NC(=O)N(Cc2ccc(F)cc2)C(=O)/C1=C/c1ccc(Br)s1] | 68.25 | 10.27 | | 62.21 | 14.85 | |
| 11 | 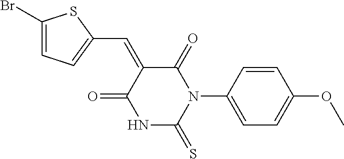 [SMILES: COc1ccc(N2C(=O)/C(=C/c3ccc(Br)s3)C(=O)NC2=S)cc1] | 47.34 | 16.16 | | 42.65 | 14.85 | |
| 12 | 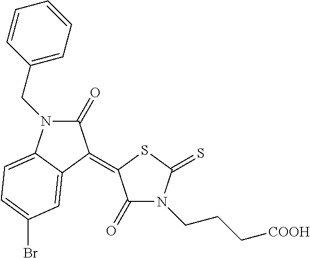 [SMILES: O=C(O)CCCN1C(=O)/C(=C2/C(=O)N(Cc3ccccc3)c3ccc(Br)cc32)SC1=S] | 74.9 | 18.82 | | 58.09 | 41.62 | |
| 13 | 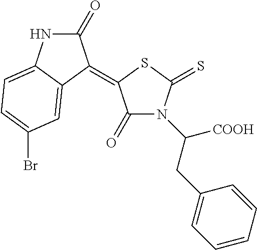 [SMILES: O=C1Nc2ccc(Br)cc2/C1=C1/SC(=S)N(C(Cc2ccccc2)C(=O)O)C1=O] | 90.11 | 10.27 | | 30.29 | −0.58 | |
| 14 | 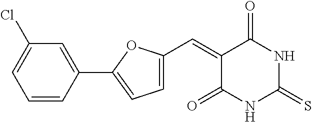 [SMILES: O=C1NC(=S)NC(=O)C1=Cc1ccc(-c2cccc(Cl)c2)o1] | 27.19 | 3.62 | | 47.79 | 7.65 | |
| 15 | 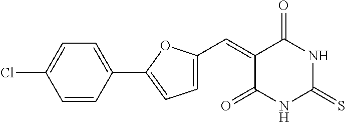 [SMILES: O=C1NC(=S)NC(=O)C1=Cc1ccc(-c2ccc(Cl)cc2)o1] | 42.21 | 3.62 | | 63.24 | 20 | |
| 16 | 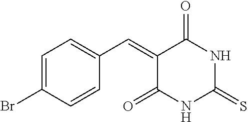 [SMILES: O=C1NC(=S)NC(=O)C1=Cc1ccc(Br)cc1] | 0 | 0 | 0 | 8.76 | 11.98 | 13.59 |
| 17 | 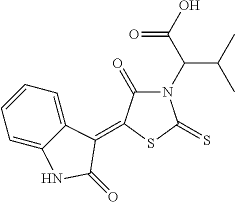 [SMILES: CC(C)C(C(=O)O)N1C(=O)/C(=C2/C(=O)Nc3ccccc32)SC1=S] | 7.03 | −2.28 | | 46.76 | 14.85 | |
| 18 | 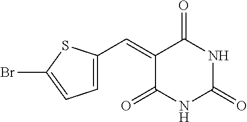 [SMILES: O=C1NC(=O)C(=Cc2ccc(Br)s2)C(=O)N1] | 0 | 0 | 0 | 18.43 | 15.2 | 13.59 |
| 19 | 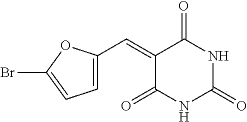 [SMILES: O=C1NC(=O)C(=Cc2ccc(Br)o2)C(=O)N1] | 12.9 | 10.68 | 3.33 | 12.57 | 2 | −1.4 |
| 20 | 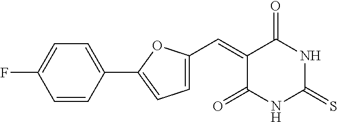 [SMILES: O=C1NC(=S)NC(=O)C1=Cc1ccc(-c2ccc(F)cc2)o1] | 28.71 | 17.11 | | 56.03 | 25.15 | |
| 21 | 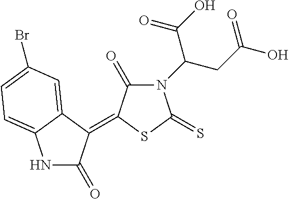 [SMILES: O=C(O)CC(C(=O)O)N1C(=O)/C(=C2/C(=O)Nc3ccc(Br)cc32)SC1=S] | 14.17 | 13.09 | 15.79 | −2.11 | −2.11 | -6.99 |
| 22 | 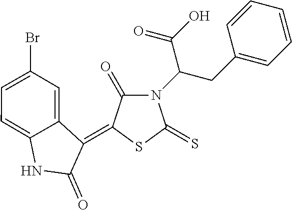 [SMILES: O=C1Nc2ccc(Br)cc2/C1=C1/SC(=S)N(C(Cc2ccccc2)C(=O)O)C1=O] | 90.11 | 83.27 | | 86.76 | 10.74 | |
| 23 | 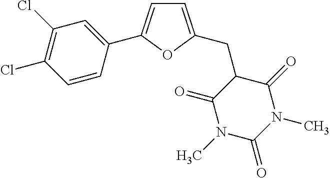 [SMILES: CN1C(=O)C(Cc2ccc(-c3ccc(Cl)c(Cl)c3)o2)C(=O)N(C)C1=O] | 10.7 | 1.54 | -8.49 | 0 | −7.35 | −8.09 |
| 24 | 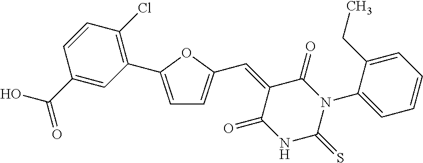 [SMILES: CCc1ccccc1N1C(=O)/C(=C/c2ccc(-c3cc(C(=O)O)ccc3Cl)o2)C(=O)NC1=S] | −10.8 | −4.63 | −10.8 | −5.88 | 2.2 | −2.94 |
| 25 | 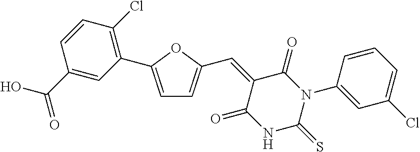 [SMILES: O=C1NC(=S)N(c2cccc(Cl)c2)C(=O)/C1=C/c1ccc(-c2cc(C(=O)O)ccc2Cl)o1] | −3.09 | −3.09 | −10.8 | −5.15 | −4.41 | −2.21 |
| 26 | 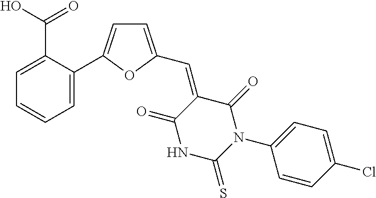 [SMILES: O=C1NC(=S)N(c2ccc(Cl)cc2)C(=O)/C1=C/c1ccc(-c2ccccc2C(=O)O)o1] | 100 | 97.02 | 2.32 | 92.44 | 16.07 | 1.47 |
| 27 | 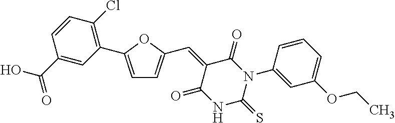 [SMILES: CCOc1cccc(N2C(=O)/C(=C/c3ccc(-c4cc(C(=O)O)ccc4Cl)o3)C(=O)NC2=S)c1] | −11.58 | 0 | −9.26 | −0.74 | 8.82 | 0.74 |
| 28 | 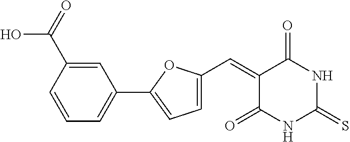 [SMILES: O=C1NC(=S)NC(=O)C1=Cc1ccc(-c2cccc(C(=O)O)c2)o1] | −13.12 | −3.09 | −9.26 | −3.68 | −2.21 | −0.74 |
| 29 | 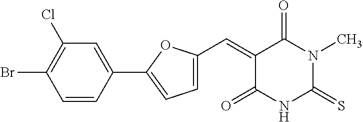 [SMILES: CN1C(=O)/C(=C/c2ccc(-c3ccc(Br)c(Cl)c3)o2)C(=O)NC1=S] | 90.11 | 80.04 | | 86.76 | 52.94 | |
| 30 | 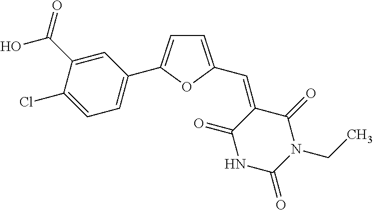 [SMILES: CCN1C(=O)NC(=O)/C(=C\c2ccc(-c3ccc(Cl)c(C(=O)O)c3)o2)C1=O] | −11.58 | 1 | −8.49 | −4.41 | −2.94 | 1.47 |

TABLE 8

| | | | | | | | | MIC, uM | |
|---|---|---|---|---|---|---|---|---|---|
| | | B. Anthracis | | | S. MRSA | | | No | 25% |
| Cmpd | Structure | 30 μM | 10 μM | 3 μM | 30 μM | 10 μM | 3 μM | Serum | Serum |
| 31 | 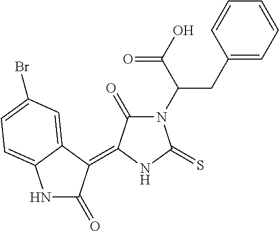 [SMILES: O=C1Nc2ccc(Br)cc2/C1=C1/NC(=S)N(C(Cc2ccccc2)C(=O)O)C1=O] | 88.02 | 2.5 | — | −2.22 | −8 | — | 250 | >250 |
| 32 | 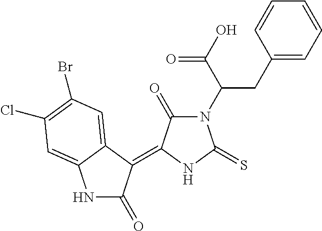 [SMILES: O=C1Nc2cc(Cl)c(Br)cc2/C1=C1/NC(=S)N(C(Cc2ccccc2)C(=O)O)C1=O] | 98.76 | 40.22 | 11.29 | 93.83 | −19.36 | −11.91 | | >250 |
| 33 | 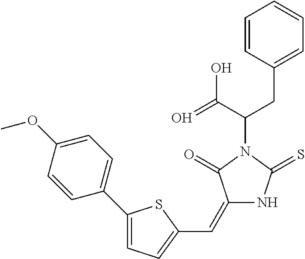 [SMILES: COc1ccc(-c2ccc(/C=C3/NC(=S)N(C(Cc4ccccc4)C(=O)O)C3=O)s2)cc1] | 100 | 0 | −3.85 | 18.49 | −13.24 | −2.94 | | |
| 34 | 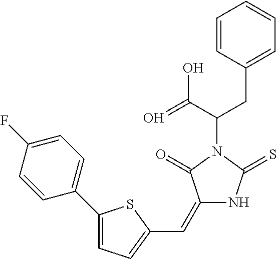 [SMILES: O=C(O)C(Cc1ccccc1)N1C(=O)/C(=C\c2ccc(-c3ccc(F)cc3)s2)NC1=S] | 98.76 | 6.99 | 0 | 46.21 | −24.79 | −15.78 | | |
| 35 | 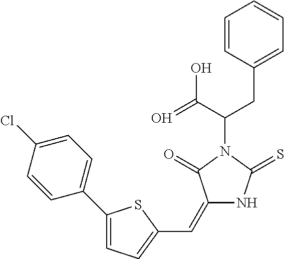 [SMILES: O=C(O)C(Cc1ccccc1)N1C(=O)/C(=C\c2ccc(-c3ccc(Cl)cc3)s2)NC1=S] | 100 | 100 | −5.4 | 90.97 | −1.47 | −2.94 | | |
| 36 | 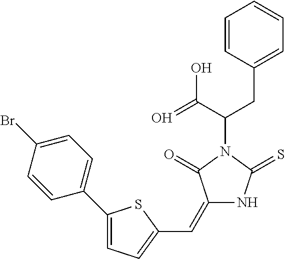 [SMILES: O=C(O)C(Cc1ccccc1)N1C(=O)/C(=C\c2ccc(-c3ccc(Br)cc3)s2)NC1=S] | 96.71 | 97.71 | 76.9 | 91.14 | 91.14 | −19.16 | | |
| 37 | 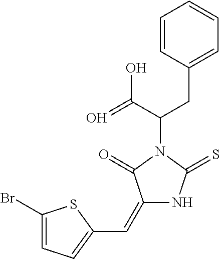 [SMILES: O=C(O)C(Cc1ccccc1)N1C(=O)/C(=C\c2ccc(Br)s2)NC1=S] | 0 | — | — | −6.27 | — | — | | |
| 38 | 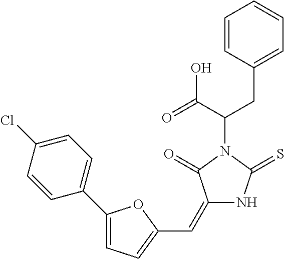 [SMILES: O=C(O)C(Cc1ccccc1)N1C(=O)/C(=C\c2ccc(-c3ccc(Cl)cc3)o2)NC1=S] | 99.71 | 68.9 | 4.99 | 86.63 | −16.9 | −13.52 | 62.5 | >250 |
| 39 | 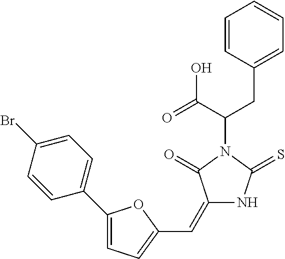 [SMILES: O=C(O)C(Cc1ccccc1)N1C(=O)/C(=C\c2ccc(-c3ccc(Br)cc3)o2)NC1=S] | 98.83 | 98.83 | 72.63 | 95.36 | 92.5 | −9.55 | | |
| 40 | 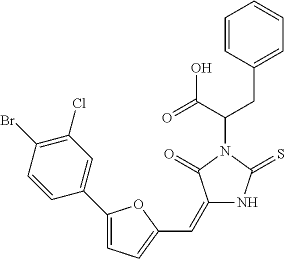 [SMILES: O=C(O)C(Cc1ccccc1)N1C(=O)/C(=C\c2ccc(-c3ccc(Br)c(Cl)c3)o2)NC1=S] | 98.08 | 98.08 | 98.08 | 95.65 | 95.65 | 17.9 | | |
| 41 | 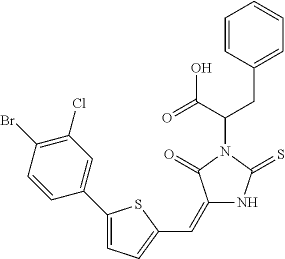 [SMILES: O=C(O)C(Cc1ccccc1)N1C(=O)/C(=C\c2ccc(-c3ccc(Br)c(Cl)c3)s2)NC1=S] | 94.47 | 8.15 | 19.46 | −0.89 | −7.45 | — | | |
| 42 | 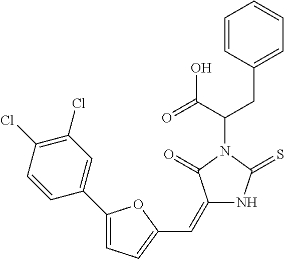 [SMILES: O=C(O)C(Cc1ccccc1)N1C(=O)/C(=C\c2ccc(-c3ccc(Cl)c(Cl)c3)o2)NC1=S] | 96.71 | 99.55 | 96.18 | 94.51 | 98.71 | −3.72 | | |
| 43 | 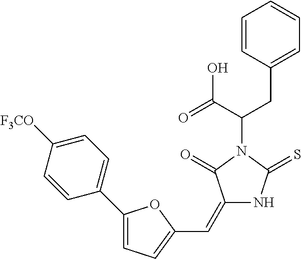 [SMILES: O=C(O)C(Cc1ccccc1)N1C(=O)/C(=C\c2ccc(-c3ccc(OC(F)(F)F)cc3)o2)NC1=S] | 96.58 | 96.69 | 8.01 | 95.15 | −6.26 | −1.32 | | |
| 44 | 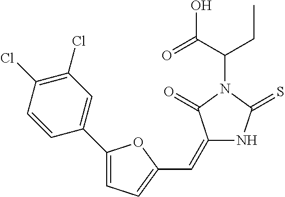 [SMILES: CCC(C(=O)O)N1C(=O)/C(=C\c2ccc(-c3ccc(Cl)c(Cl)c3)o2)NC1=S] | 93.95 | 97.18 | 4.13 | 92.9 | 4.53 | −2.47 | 250 | >250 |
| 45 | 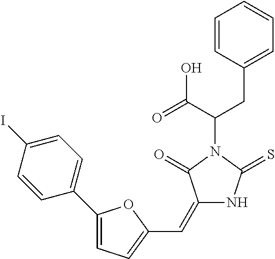 [SMILES: O=C(O)C(Cc1ccccc1)N1C(=O)/C(=C\c2ccc(-c3ccc(I)cc3)o2)NC1=S] | 96.38 | 97.44 | 49.17 | 92.68 | 88.21 | −1.84 | | |
| 46 | 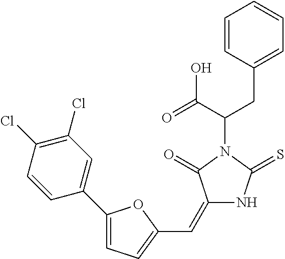 [SMILES: O=C(O)C(Cc1ccccc1)N1C(=O)/C(=C\c2ccc(-c3ccc(Cl)c(Cl)c3)o2)NC1=S] | 106.96 | 108.46 | 108.4 | 103.37 | 93.71 | −17.39 | 15.6 | >250 |
| 47 | 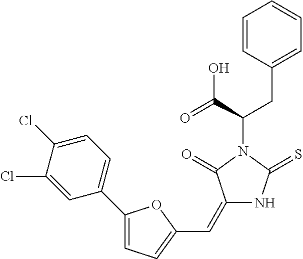 [SMILES: O=C(O)[C@@H](Cc1ccccc1)N1C(=O)/C(=C\c2ccc(-c3ccc(Cl)c(Cl)c3)o2)NC1=S] | 106.64 | 107.46 | 9.41 | 102.93 | 84.4 | −13.75 | 7.8 | 250 |
| 48 | 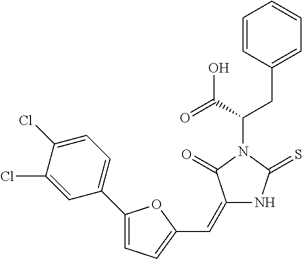 [SMILES: O=C(O)[C@H](Cc1ccccc1)N1C(=O)/C(=C\c2ccc(-c3ccc(Cl)c(Cl)c3)o2)NC1=S] | 106.88 | 108.18 | −0.39 | 103.62 | 80.58 | −16.05 | 7.8 | 250 |
| 49 | 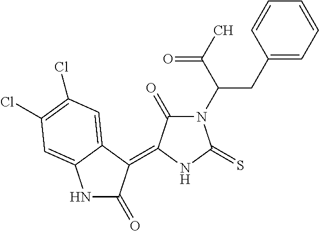 [SMILES: CC(=O)C(Cc1ccccc1)N1C(=O)/C(=C2/C(=O)Nc3cc(Cl)c(Cl)cc32)NC1=S] | 97.21 | −7.49 | 1.73 | −26.32 | −20.3 | −7.73 | | |
| 50 | 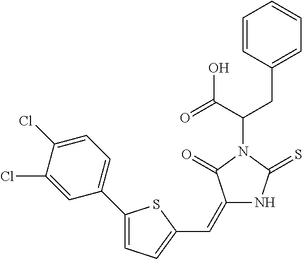 [SMILES: O=C(O)C(Cc1ccccc1)N1C(=O)/C(=C\c2ccc(-c3ccc(Cl)c(Cl)c3)s2)NC1=S] | 94.17 | 107.59 | 1.03 | 35.6 | 14.28 | −14.58 | | |
| 51 | 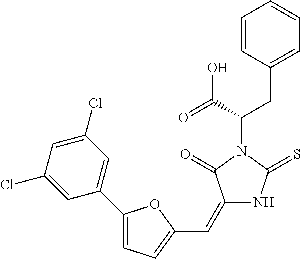 [SMILES: O=C(O)[C@H](Cc1ccccc1)N1C(=O)/C(=C\c2ccc(-c3cc(Cl)cc(Cl)c3)o2)NC1=S] | 99.3 | −3.08 | −0.16 | 85.33 | −7.03 | −0.61 | | |
| 52 | 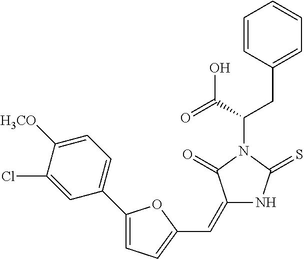 [SMILES: COc1ccc(-c2ccc(/C=C3/NC(=S)N([C@@H](Cc4ccccc4)C(=O)O)C3=O)o2)cc1Cl] | 95.53 | 27.26 | −5.68 | 82.2 | −11.68 | −7.99 | 62.5 | >250 |
| 53 | 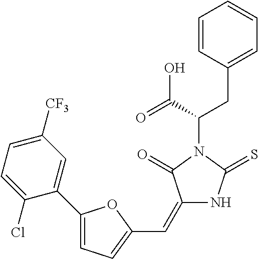 [SMILES: O=C(O)[C@H](Cc1ccccc1)N1C(=O)/C(=C\c2ccc(-c3cc(C(F)(F)F)ccc3Cl)o2)NC1=S] | 92.5 | 95.5 | 88.24 | 92.88 | 92.3 | −4.72 | 15.6 | >250 |
| 54 | 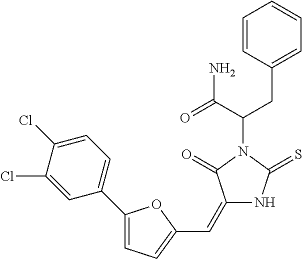 [SMILES: NC(=O)C(Cc1ccccc1)N1C(=O)/C(=C\c2ccc(-c3ccc(Cl)c(Cl)c3)o2)NC1=S] | 92.96 | 94.76 | 20.56 | 57.52 | 30.05 | 2.03 | >250 | >250 |
| 55 | 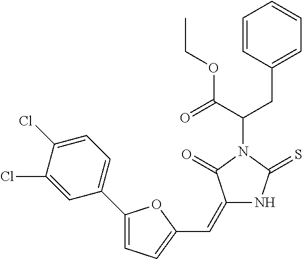 [SMILES: CCOC(=O)C(Cc1ccccc1)N1C(=O)/C(=C\c2ccc(-c3ccc(Cl)c(Cl)c3)o2)NC1=S] | -6.41 | -50.84 | — | −2.92 | 0.57 | — | >250 | >250 |
| 56 | 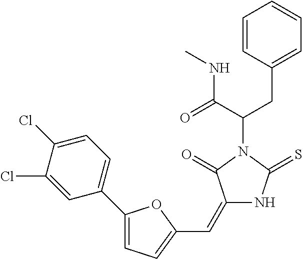 [SMILES: CNC(=O)C(Cc1ccccc1)N1C(=O)/C(=C\c2ccc(-c3ccc(Cl)c(Cl)c3)o2)NC1=S] | | | | | | | >250 | >250 |
| 57 | 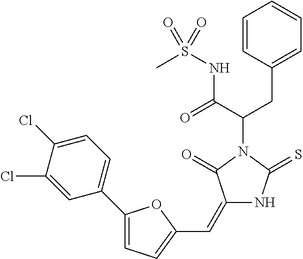 [SMILES: CS(=O)(=O)NC(=O)C(Cc1ccccc1)N1C(=O)/C(=C\c2ccc(-c3ccc(Cl)c(Cl)c3)o2)NC1=S] | 90.24 | 96.38 | 45.19 | 58.02 | 30.87 | 3.18 | 15.6 | >250 |
| 58 | 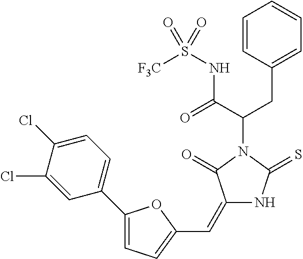 [SMILES: O=C(NS(=O)(=O)C(F)(F)F)C(Cc1ccccc1)N1C(=O)/C(=C\c2ccc(-c3ccc(Cl)c(Cl)c3)o2)NC1=S] | 97.43 | 98.67 | 69.4 | 94.55 | 81.66 | 0.23 | 7.8 | >250 |
| 59 | 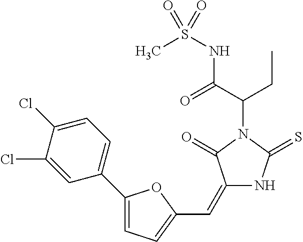 [SMILES: CCC(C(=O)NS(C)(=O)=O)N1C(=O)/C(=C\c2ccc(-c3ccc(Cl)c(Cl)c3)o2)NC1=S] | 19.58 | 6.23 | 6.4 | 0.06 | 2.56 | 1.71 | 125 | >250 |
| 60 | 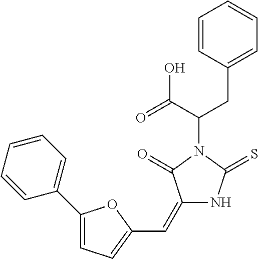 [SMILES: O=C(O)C(Cc1ccccc1)N1C(=O)/C(=C\c2ccc(-c3ccccc3)o2)NC1=S] | 18 | 11.62 | 2.29 | -8.8 | −5.71 | −2.55 | 125 | >250 |
| 61 | 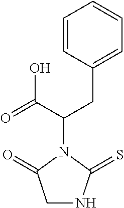 [SMILES: O=C(O)C(Cc1ccccc1)N1C(=O)CNC1=S] | | | | | | | >250 | >250 |
| 62 | 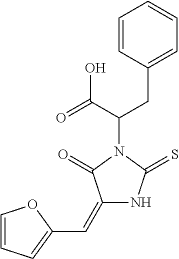 [SMILES: O=C(O)C(Cc1ccccc1)N1C(=O)/C(=C\c2ccco2)NC1=S] | | | | | | | >250 | >250 |
| 63 | 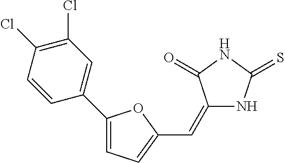 [SMILES: O=C1NC(=S)N/C1=C/c1ccc(-c2ccc(Cl)c(Cl)c2)o1] | | | | | | | 62.5 | >250 |
| 64 | 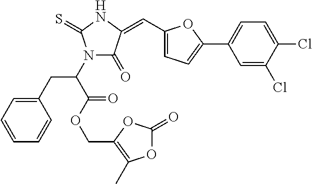 [SMILES: Cc1oc(=O)oc1COC(=O)C(Cc1ccccc1)N1C(=O)/C(=C\c2ccc(-c3ccc(Cl)c(Cl)c3)o2)NC1=S] | −1.34 | 0.22 | −0.01 | −3.34 | −1.48 | 0.71 | >250 | >250 |
| 65 | 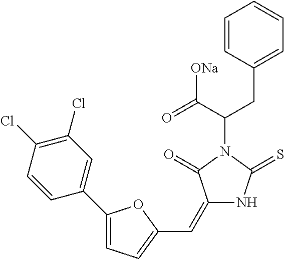 | 104.01 | 107.86 | 108.67 | 105.68 | 104.72 | 0.9 | 7.8 | >250 |
| 66 | 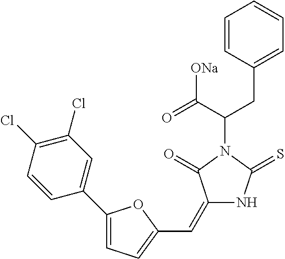 | 104.01 | 107.86 | 108.67 | 105.68 | 104.72 | 0.9 | 7.8 | >250 |
| 67 | 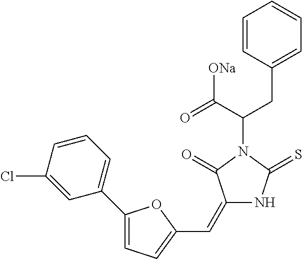 | 87.61 | 4.51 | 6.01 | −7.41 | −4.15 | 0.37 | 250 | >250 |
| 68 | 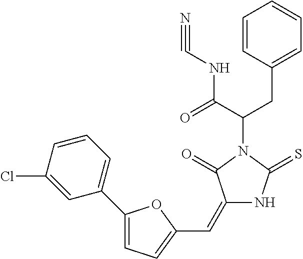 [SMILES: N#CNC(=O)C(Cc1ccccc1)N1C(=O)/C(=C\c2ccc(-c3cccc(Cl)c3)o2)NC1=S] | 12.53 | 6.31 | −1.9 | −0.93 | −1.1 | −0.24 | 125 | >250 |
| 69 | 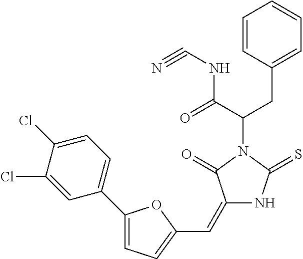 [SMILES: N#CNC(=O)C(Cc1ccccc1)N1C(=O)/C(=C\c2ccc(-c3ccc(Cl)c(Cl)c3)o2)NC1=S] | 99.2 | 99.95 | 0.13 | 97.49 | 0.65 | 0.03 | 15.6 | 250 |
| 70 | 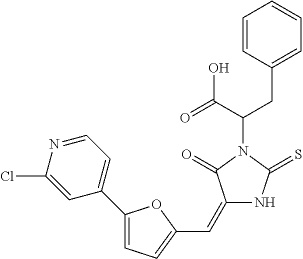 [SMILES: O=C(O)C(Cc1ccccc1)N1C(=O)/C(=C\c2ccc(-c3ccnc(Cl)c3)o2)NC1=S] | 4.62 | 15.98 | 2.54 | −1.4 | −1.26 | −0.84 | 125 | >250 |
| 71 | 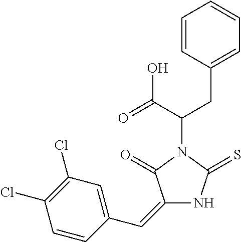 [SMILES: O=C(O)C(Cc1ccccc1)N1C(=O)/C(=C\c2ccc(Cl)c(Cl)c2)NC1=S] | −17.31 | −8.93 | 0.05 | −5.12 | −2.74 | −0.86 | >250 | >250 |
| 72 | 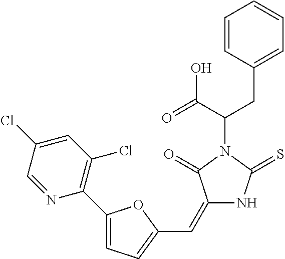 [SMILES: O=C(O)C(Cc1ccccc1)N1C(=O)/C(=C\c2ccc(-c3ncc(Cl)cc3Cl)o2)NC1=S] | 98.08 | 9.07 | −13.47 | 52.99 | −5.02 | −2.17 | 62.5 | 250 |
| 73 | 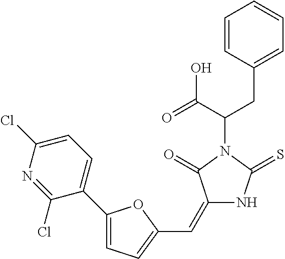 [SMILES: O=C(O)C(Cc1ccccc1)N1C(=O)/C(=C\c2ccc(-c3ccc(Cl)nc3Cl)o2)NC1=S] | | | | | | | 125 | >250 |
| 74 | 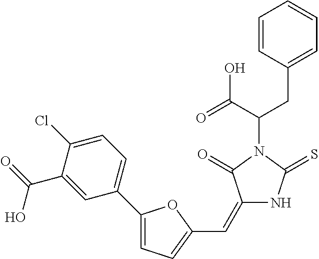 [SMILES: O=C(O)c1cc(-c2ccc(/C=C3/NC(=S)N(C(Cc4ccccc4)C(=O)O)C3=O)o2)ccc1Cl] | | | | | | | >250 | >250 |
| 75 | 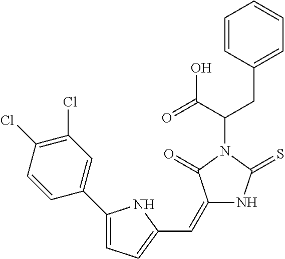 [SMILES: O=C(O)C(Cc1ccccc1)N1C(=O)/C(=C\c2ccc(-c3ccc(Cl)c(Cl)c3)[nH]2)NC1=S] | | | | | | | 62.5 | >250 |
| 76 | 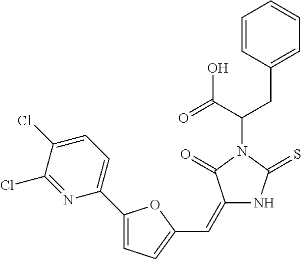 [SMILES: O=C(O)C(Cc1ccccc1)N1C(=O)/C(=C\c2ccc(-c3ccc(Cl)c(Cl)n3)o2)NC1=S] | | | | | | | 62.5 | >250 |
| 77 | 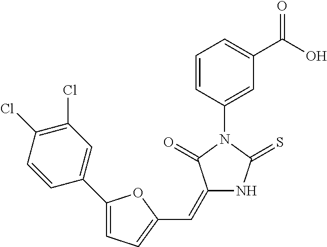 [SMILES: O=C(O)c1cccc(N2C(=O)/C(=C\c3ccc(-c4ccc(Cl)c(Cl)c4)o3)NC2=S)c1] | | | | | | | 31.3 | >250 |
| 78 | 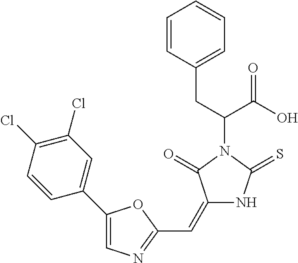 [SMILES: O=C(O)C(Cc1ccccc1)N1C(=O)/C(=C\c2ncc(-c3ccc(Cl)c(Cl)c3)o2)NC1=S] | | | | | | | 250 | >250 |
| 79 | 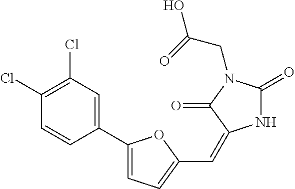 [SMILES: O=C(O)CN1C(=O)N/C(=C/c2ccc(-c3ccc(Cl)c(Cl)c3)o2)C1=O] | | | | | | | >250 | >250 |
| 80 | 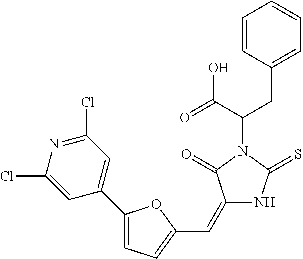 [SMILES: O=C(O)C(Cc1ccccc1)N1C(=O)/C(=C\c2ccc(-c3cc(Cl)nc(Cl)c3)o2)NC1=S] | | | | | | | 62.5 | >250 |
| 81 | 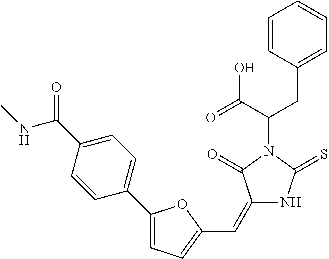 [SMILES: CNC(=O)c1ccc(-c2ccc(/C=C3/NC(=S)N(C(Cc4ccccc4)C(=O)O)C3=O)o2)cc1] | | | | | | | >250 | >250 |
| 82 | 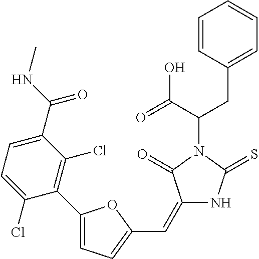 [SMILES: CNC(=O)c1ccc(Cl)c(-c2ccc(/C=C3/NC(=S)N(C(Cc4ccccc4)C(=O)O)C3=O)o2)c1Cl] | | | | | | | >250 | >250 |
| 83 | 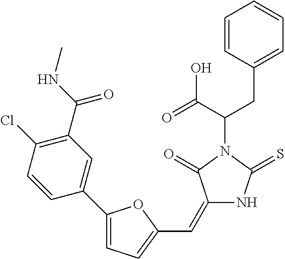 [SMILES: CNC(=O)c1cc(-c2ccc(/C=C3/NC(=S)N(C(Cc4ccccc4)C(=O)O)C3=O)o2)ccc1Cl] | | | | | | | >250 | >250 |
| 84 | 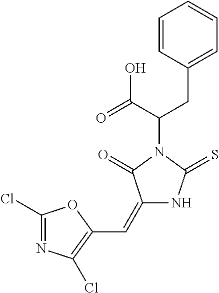 [SMILES: O=C(O)C(Cc1ccccc1)N1C(=O)/C(=C\c2oc(Cl)nc2Cl)NC1=S] | | | | | | | >250 | >250 |
| 85 | 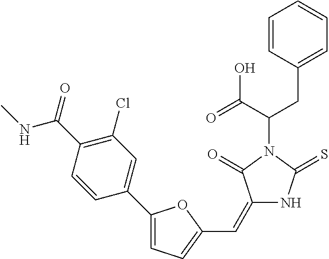 [SMILES: CNC(=O)c1ccc(-c2ccc(/C=C3/NC(=S)N(C(Cc4ccccc4)C(=O)O)C3=O)o2)cc1Cl] | | | | | | | >250 | >250 |
| 86 | 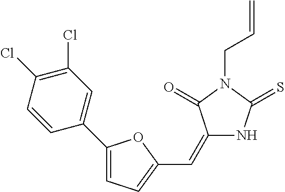 [SMILES: C=CCN1C(=O)/C(=C\c2ccc(-c3ccc(Cl)c(Cl)c3)o2)NC1=S] | | | | | | | >250 | >250 |
| 87 | 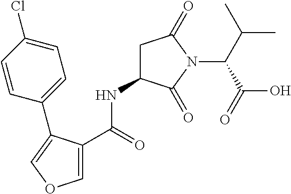 [SMILES: CC(C)[C@H](C(=O)O)N1C(=O)C[C@H](NC(=O)c2cocc2-c2ccc(Cl)cc2)C1=O] | | | | | | | >250 | >250 |
| 88 | 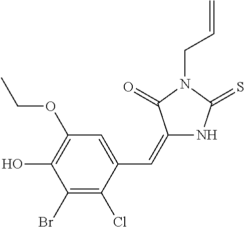 [SMILES: C=CCN1C(=O)/C(=C\c2cc(OCC)c(O)c(Br)c2Cl)NC1=S] | | | | | | | >250 | >250 |
| 89 | 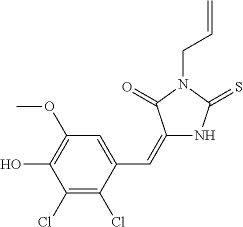 [SMILES: C=CCN1C(=O)/C(=C\c2cc(OC)c(O)c(Cl)c2Cl)NC1=S] | | | | | | | 250 | >250 |
| 90 | 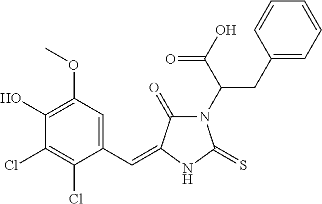 [SMILES: COc1cc(/C=C2/NC(=S)N(C(Cc3ccccc3)C(=O)O)C2=O)c(Cl)c(Cl)c1O] | | | | | | | >250 | >250 |
| 91 | 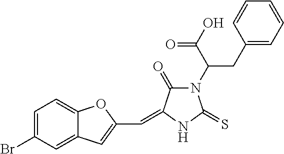 [SMILES: O=C(O)C(Cc1ccccc1)N1C(=O)/C(=C\c2cc3cc(Br)ccc3o2)NC1=S] | | | | | | | 125 | >250 |
| 92 | 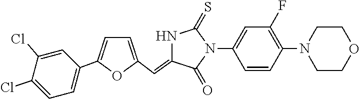 [SMILES: O=C1/C(=C/c2ccc(-c3ccc(Cl)c(Cl)c3)o2)NC(=S)N1c1ccc(N2CCOCC2)c(F)c1] | | | | | | | | |

TABLE 9

| Organism | MIC* |
|---|---|
| B. anthracis Sterne | 4.0 μg ml⁻¹ (7.6 μM) |
| B. anthracis ΔSterne | 4.0 μg ml⁻¹ (7.6 μM) |
| S. aureus RN4220 | 8.0 μg ml⁻¹ (16.0 μM) |

*Determined using the microbroth dilution method. The MIC is the amount of drug needed to prevent growth of 5 × 10⁵ bacteria suspended in 0.1 ml nutrient broth and incubated in a 96-well microtiter plate at 37° C. for 24 hours.
*Determined using agar dilution method.

Several lines of evidence indicate that Epimerox specifically binds to and inhibits 2-epimerase. Firstly, Epimerox was identified by in vitro docking with the active and allosteric sites of BA5509; the only homologous protein in B. anthracis is BA5433. Secondly, phenotypic similarities between the RS1205 2-epimerase mutant and Epimerox-treated cells, defined by bulging filaments, aberrant septation, and reduced GFP-PlyG^{BD} binding are striking Thirdly, by increasing the concentration of IPTG in the growth medium of the BA5509 or the BA5509 BA5433 mutant strains (each bearing P^{SPAC}-BA5509) from 0.1 mM to 25 mM, the MIC (MIC=minimal inhibitory concentration) for Epimerox increased from 4.0 μm1⁻¹ to 8 μm1⁻¹ (Table 10). The 2-fold increase in Epimerox MIC was associated with a 10- and 20-fold increase in 2-epimerase expression in the BA5509 BA5433 and BA5509 mutants, respectively. Although these findings show a correlation between increased Epimerox resistance and BA5509 expression and strongly suggest a direct interaction between Epimerox the inhibitor and 2-epimerase, secondary targets (i.e., other than 2-epimerase) for Epimerox in B. anthracis cannot be ruled out.

TABLE 10

| IPTG | BA5509 BA5433 mutant | BA5509 mutant | Epimerox MIC |
|---|---|---|---|
| (mM) | log2(exp/ref)** | log2(exp/ref) | (μg/ml) |
| 0.01 | −0.25 | −0.10 | 4 |
| 0.05 | 0.36 | −0.46 | 4 |
| 0.1 | 1.82 | 0.51 | 4 |
| 0.25 | 1.42 | 0.35 | 4 |
| 0.5 | 1.73 | 3.19 | 4 |
| 1 | 2.79 | 3.84 | 8 |
| 5 | 2.76 | 4.00 | 8 |
| 10 | 3.09 | 4.45 | 8 |
| 25 | 3.20 | 4.39 | 8 |

*Two sets of the 2-epimerase single mutant (BA5509) and double mutant (BA5509 BA5433), each bearing the IPTG-inducible P^{SPAC}-BA5509 fusion, were grown in the presence of a range of indicated IPTG concentrations. One set was used to determine the MIC of Epimerox according to the standard broth microdilution method¹. The second set was grown for 5 hours prior to the extraction of RNA and processing for qRT-PCR analysis in the manner described⁴⁰.

Although Epimerox was constructed to target the B. anthracis 2-epimerase, it nonetheless inhibited the growth of many other Gram-positive organisms that also encode 2-epimerases (FIG. 9d). Staphylococcus aureus strain RN4220, in particular, did not grow over a 12 hour period in the presence of Epimerox concentrations above 7.5 μm1⁻¹ (FIG. 9e). At 7.5 μg m1⁻¹ (14 μM), S. aureus grew very poorly and manifested cell division defects, including aberrant septa positioning and excessive cell wall material (FIG. 9c). These results were identical to those previously observed with S. aureus CWG mutants. (14) Since the S. aureus 2-epimerase differs from that of B. anthracis in the number of amino acids predicted to make contact with Epimerox (5 of 12 contact amino acids differ in the S. aureus epimerase), the superior activity of Epimerox against B. anthracis is not surprising. The activity against S. aureus does, however, indicate the potential for developing antibacterial molecules that can target bacterial 2-epimerases.

### Example 3

### Validation of Antimicrobial Target In Vivo

Based on the potent in vitro activity of Epimerox against B. anthracis, its activity was tested in a mouse model in which animals were infected with bacteria through intraperitoneal (i.p.) administration. Although this route of infection does not reflect the natural biological route of infection by B. anthracis (or a likely route for antibiotic delivery in clinical settings), it is, nonetheless, a reliable and well-recognized model that has been used in several studies assessing the efficacy of lysins as antibacterial agents. (7, 8, 22).

Overnight B. anthracis Sterne cultures were diluted 1:100 in BHI and grown for 3 h with aeration at 30° C. Cells were harvested, washed with sterile PBS (pH 7.2), adjusted to a density of 1×10⁶ cell per ml of PBS, and plated onto nutrient agar plates after appropriate dilution. Four to six week-old female C57BL/6 mice (fifteen per group) were then infected i.p. with 5×10⁵ bacilli. (7) Starting at either 3 or 24 hours post-infection, Epimerox was administered i.p. every six hours for up to seven days at dosages of either 20 ng (1.3 mg/kg) or 200 μg (13 mg/kg). Survival was monitored for 14 days. A second set of infected mice also was euthanized at indicated time points for necropsy. Heart, liver, spleen, and kidneys were excised, washed with 70% ethanol and sterile PBS (pH 7.2), homogenized in PBS, and plated to determine the number of viable bacteria in the various tissues. Uninfected mice were used to confirm the sterility of each organ.

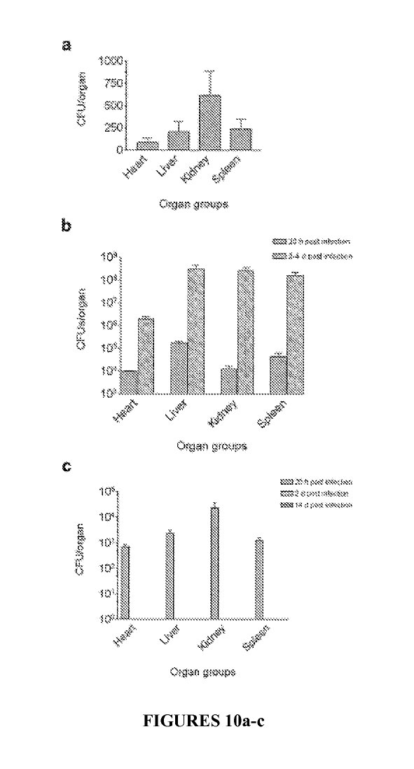

In this model system, and in the absence of Epimerox treatment, major organs became colonized at 3 hours post-infection with 5×10⁵ vegetative B. anthracis bacilli, and death of all animals was observed at 5 days and 50% survival was seen at about 3 days (FIG. 9f, FIGS. 10a and 10b). FIGS. 10a-c show the bacterial load in Epimerox treated and untreated mice. (a) Bacteria were detected in mouse organs three hours after i.p. infection. Three mice were euthanized at 3 h and the indicated organs were removed to determine the number of colony forming units per organ. Mean values with s.d. are shown. (b) The bacterial load in mice treated with buffer at 3 h post-infection. Samples at 20 h were taken from euthanized mice, while samples at 2-4 d were taken after death from infection. (c) The bacterial load in mice treated with Epimerox at 3 h post-infection. Animals were euthanized at 20 h, 2 d, and 14 d post-infection and processed for organ recovery and bacterial titer determination. When Epimerox was administered i.p. at either 3 or 24 hours post-infection (and continued every 6 hours for 7 days), 100% and 66% of the animals, respectively, survived 14 days. When mice were treated with Epimerox at 3 hours post-infection, B. anthracis was detected in the major organs at 20 hours, but was not observed after 2 days (FIG. 10c). These experiments indicated that a course of Epimerox therapy was capable of rescuing animals that had been infected with B. anthracis.

### Example 4

### Testing the Ability of the Antimicrobial Target to Develop Resistance

Epimerox was compared with other antibiotic compounds to evaluate whether, and to what degree, bacteria challenged with the antibiotic would develop resistance to the drug. Minimal inhibitory concentrations (MICs) were determined using the microbroth-dilution method as described. (32). For analysis of the level of drug resistance that could be induced by Epimerox, rifampin (Sigma-Aldrich), and daptomycin (Tocris Bioscience), various cell strains were grown in 100 ml BHI with agitation at 30° C. (B. anthracis Sterne and derivatives thereof) or 37° C. (S. aureus RN4220). After 24 hours, cultures were washed, concentrated 10-fold in media, and plated for viability on agar with or without daptomycin (15 ug m1⁻¹), rifampin (50 μm1⁻¹), or a range of Epimerox concentrations. Where indicated, IPTG (at various concentrations) was added to both growth cultures and agar plates. Colonies appearing after 3-5 days were used to calculate resistance frequency.

Induction of Epimerox resistance was assessed using serial passage in a manner similar to that described previously. (23) Briefly, overnight cultures of B. anthracis ΔSterne and S. aureus RN4220 were adjusted to OD₆₀₀ 0.1 in BHI medium with Epimerox (ranging in concentration from 1 to 15 μM in 1 μM increments) and grown for 18 h at 30° C. with aeration. The highest Epimerox concentration that yielded visible growth was washed in PBS, adjusted to OD₆₀₀ 0.1, and aliquots were either frozen at −80° C. for later analysis or incubated overnight with a range of increasing Epimerox concentrations as above. Although resistance values did not increase after 6 days, the experiment was ultimately continued for 21 days. After 21 days, all frozen intermediaries were revived, subcultured three times in the absence of Epimerox, and the MIC of Epimerox was again determined by broth microdilution. Similar experiments were performed with daptomycin and S. aureus strain RN4220.

The attractiveness of Epimerox as a lead molecule for antimicrobial drug development is reinforced by the observation that resistance to its activity (as with PlyG) appears to be below normally detectable levels. Considering, however, the effect of increased BA5509 expression on the MIC of Epimerox (Table 1) and the potential influence of changes in gene dosage, a formal analysis of resistance to Epimerox was undertaken. First, the appearance of spontaneous mutants resistant to Epimerox was analyzed using rifampin-treated and daptomycin-treated bacteria as controls. The frequency of induction of resistance to rifampin and daptomycin was observed to be between 10⁻⁷ and 10⁻⁹ in B. anthracis and S. aureus respectively (Table 9).

TABLE 9
Spontaneous antimicrobial resistance.

| Treatment* | Organism** | Frequency of resistance*** |
|---|---|---|
| rifampin | B. anthracis | 3.0 × 10⁻⁹ |
| (50 μg ml⁻¹) | S. aureus | 7.7 × 10⁻⁷ |
| daptomycin | B. anthracis | 1.5 × 10⁻⁷ |
| (15 μg ml⁻¹) | S. aureus | 1.9 × 10⁻⁹ |
| Epimerox | B. anthracis (3 μM) | <8.3 × 10⁻¹¹ |
| | S. aureus (10 μM) | <4.5 × 10⁻¹¹ |

*Concentrated cultures were plated to agar with the indicated treatments.
**B. anthracis Sterne and S. aureus RN4220 were used in this study.
***Epimerox-resistant colonies were not observed in any experiment.

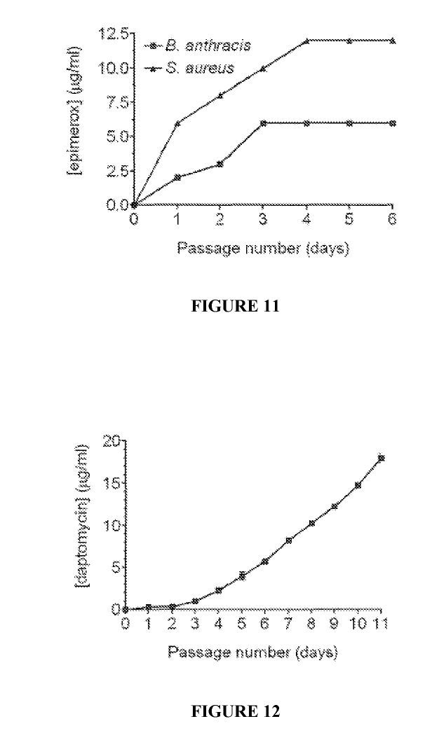

FIG. 11 shows the Epimerox serial passage experiments. The highest concentration of Epimerox (in μg/ml) yielding growth is shown for each day of passage. No further increases were observed after six days. Squares, B. anthracis Sterne; triangles, S. aureus RN4220. FIG. 12 shows the Daptomycin serial passage experiment. The highest concentration of daptomycin (in μg/ml) yielding growth of S. aureus strain RN4220 is shown for each day of passage. In contrast, repeated inoculations of each bacterial strain onto media supplemented with Epimerox at or near MIC values, failed to yield resistant derivatives. Efforts then were undertaken to generate Epimerox-resistance using the serial passage method described for S. aureus. (23) After six days, the Epimerox MIC plateaued at 8.0 μg m1⁻¹ for B. anthracis and 12 μg m1⁻¹ for S. aureus from starting MICs of 2.0 ng m1⁻¹ and 5.0 μg m1⁻¹, respectively (FIG. 11). Further increases were never observed (up to 21 days). Despite the slight increase in Epimerox MIC (perhaps driven by an increase in BA 5509 expression in B. anthracis), high-level resistance similar to that observed with daptomycin by Palmer et al. (23) and here (involving an increase from 0.5 μm1⁻¹ to 18 μm1⁻¹ in only 11 days; FIG. 12), was not observed. These results support the described approach for identifying a novel antimicrobial target with reduced potential for resistance, as well as antimicrobial agents that advantageously interfere with the target.

### Example 5

### Comparison of the 2-Epimerase Target in Other Microorganisms

A. Comparison of 2-Epimerase in Other Gram-Positive Bacteria.

The bacterial 2-epimerase catalyzes the reversible conversion of UDP-N-acetylglucosaminec (UDP-GlcNAc) and UDP-N-acetylmannosamine (UDP-ManNAc). 2-epimerase provides bacteria with the activated form of ManNAc found in the linkage unit that serves to attach teichoic acids to the peptidoglycan in Gram-positive bacteria. ManNAc residues are found as components of the enterobacterial common antigen (ECA), a surface antigen found in all enteric or gut bacteria.

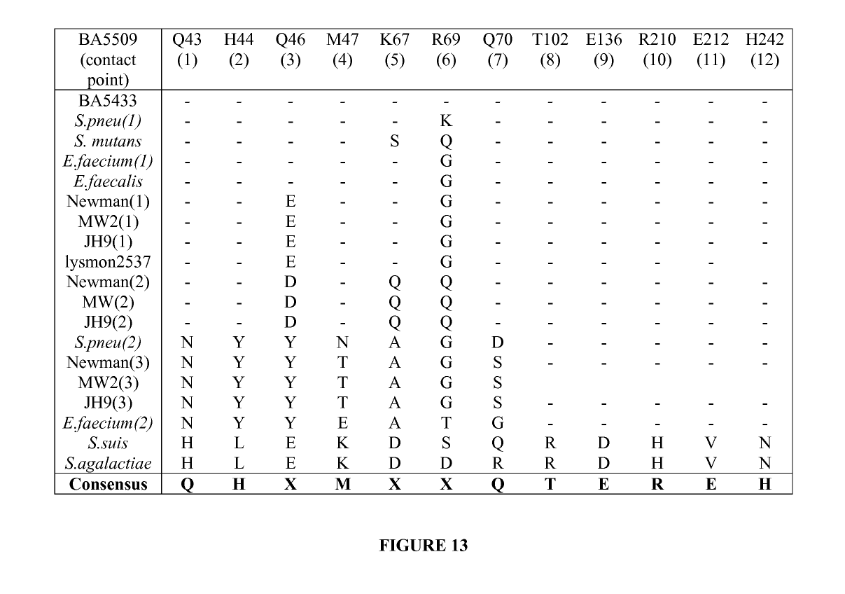

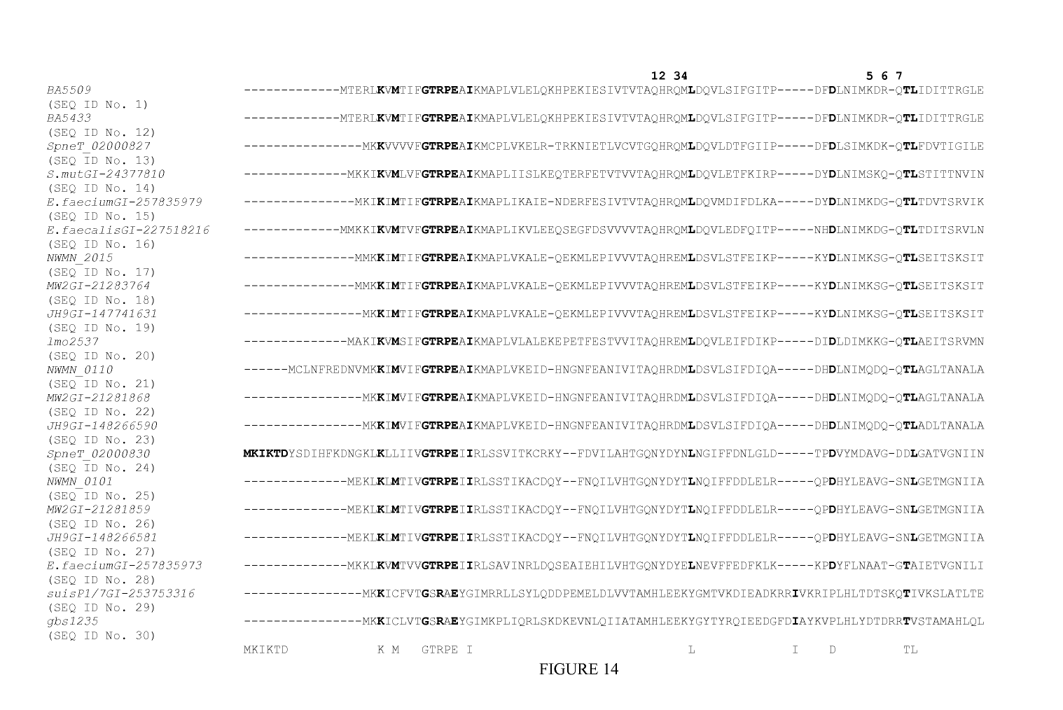

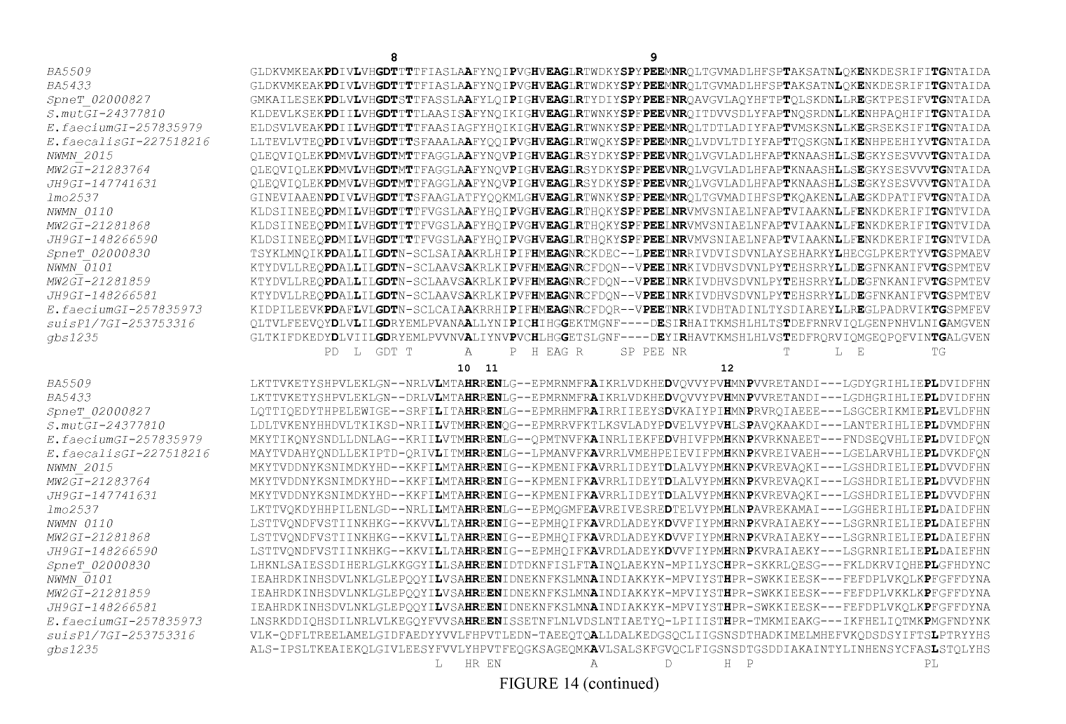

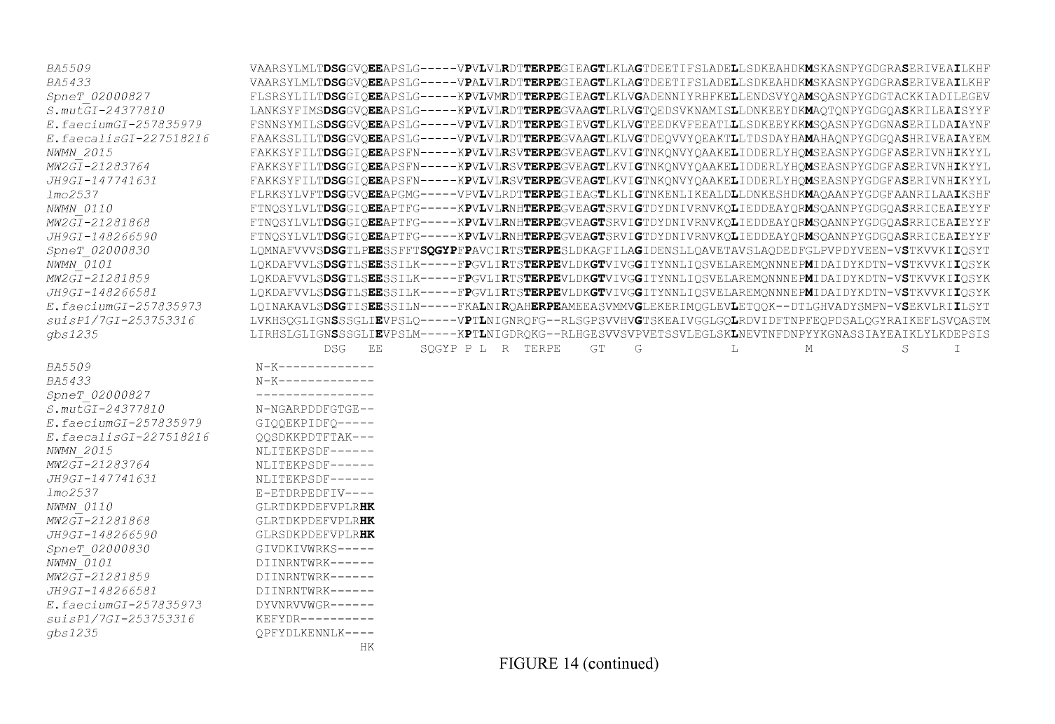

A BLAST analysis of the B. anthracis 2-epimerase with the genome of other Gram-positive bacteria was performed, and the results shown in FIGS. 13 and 14. FIG. 13 shows the 12 amino acitds of the allosteric pocket in Bacillus anthracis Sterne: BA5509, BA5433; Staphylococcus aureus Newman: NWMN 2015 (1), NWMN_{—}0110(2), NWMN_{—}0101 (3); Staphylococcus aureus MW2: MW2GI-21283764 (1), MW2GI-21281868 (2), MW2GI-21281859 (3); Staphylococcus aureus JH9: JH9GI-147741631(1), JH9GI-148266590 (2), JH9GI-148266581 (3); Enterococcus faecium Com15: GI-257835979 (1), GI-257835973 (2); Enterococcus faecalis TX0104: GI-227518216; Streptococcus pneumoniae TIGR4: SpneT_{—}02000827 (1), SpneT_{—}02000827 (2); Streptococcus agalactiae NEM316: gbs1235; Streptococcus mutans U159: GI-24377810; Streptococcus suis P1/7: GI-253753316; Listeria monocytogenes: lmo2537. FIG. 14 shows the alignments for amino acid sequences of bacterial 2-epimerase for the Gram-positive bacteria described in FIG. 13, including SEQ ID Nos. 12-30. Of the 12 amino acids in the allosteric binding pocket that were shown previously to contact UDP-GlcNAc, several bacterial species showed high sequence homology (FIG. 13). All strains of Staphylococci, S. pneumoniae, S. mutans, E. faecalis, E. faecium, and Listeria monocytogenes had the highest homology with the 2-epimerases of B. anthracis. Of the genomes with more than one 2-epimerase, at least one had the highest homology. The consensus sequence of the 12 contact points among the aligned sequences was: QHXMXXQTEREH (FIGS. 13, 14).

B. Comparison of 2-Epimerase in Gram-Negative Bacteria.

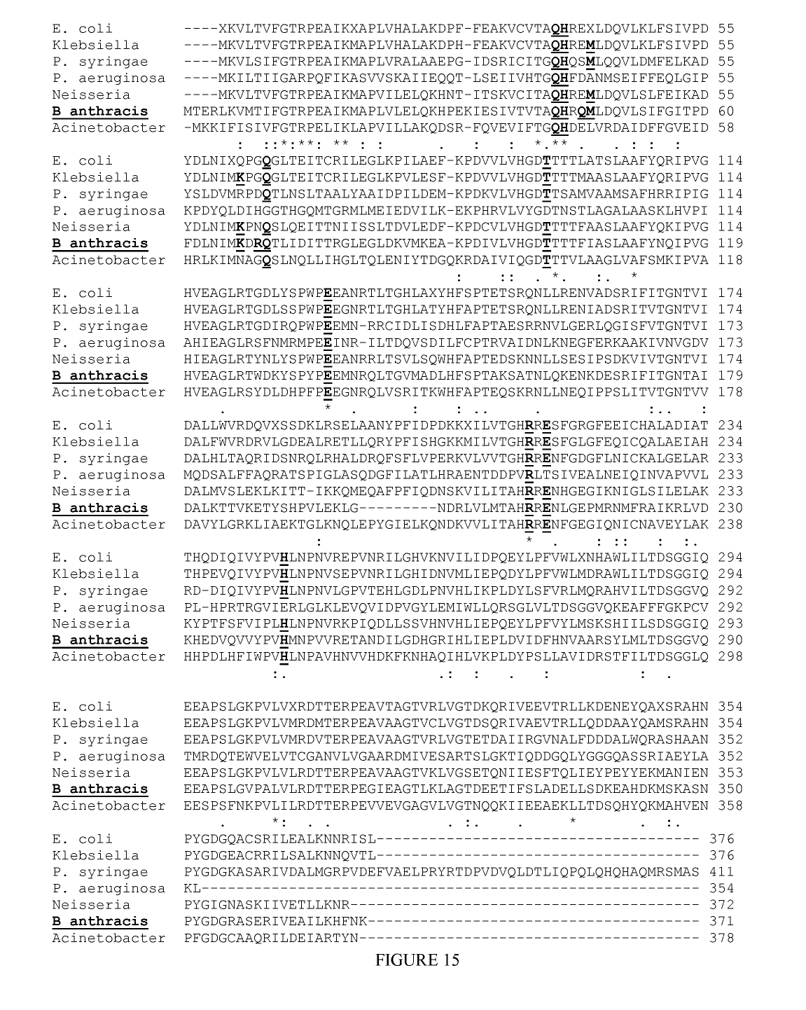

A BLAST analysis of the B. anthracis 2-epimerase with the genome of Gram-negative bacteria 2-epimerase (Neisseria meningitis, E. coli, Pseudomonas syringae, Klebsiella pneumoniae, Aninetobacter baumanii) also was conducted. FIG. 15 shows the alignment of B. anthracis 2-epimerase with the genome of Gram-negative bacteria 2-epimerase for: E. coli (SEQ ID No. 31), Klebsiella pneumoniae (SEQ ID No. 32), Pseudomonas syringae (SEQ ID No. 33), P. Pseudomonas aeruginosa (SEQ ID No. 34), Neisseria meningitis (SEQ. ID No. 35), Acinetobacter baumanii, (SEQ ID No. 36). Bold homologies are the 12 amino acids found in the allosteric site contact points for UDP-Nacetylglucosamine. Consensus sequence (QHXXXQTEREH without P. aeruginosa and QHXXXXERXX with P. aeruginosa). As with the Gram-positive 2-epimerase, high homology was observed. For the 12 amino acids in the allosteric binding pocket that made contact with the (UDP-GlcNAc), all of the Gram-negative species examined had homology with the 2-epimerases of B. anthracis. The consensus sequence of the 12 contact points was: QHXXXQTEREH if P. aeruginosa was not included and QHXXXXERXX when P. aeruginosa was included in the comparison.

To facilitate an understanding of the principles and features of the invention, various illustrative embodiments are described in this specification. Although exemplary embodiments are explained in detail, it is to be understood that other embodiments are contemplated. Accordingly, neither the invention, nor any of the appended claims, is limited in its scope to specific examples or embodiments herein, or to the details of construction and arrangement of components set forth in the foregoing description or illustrated in the drawings. The invention is capable of other embodiments and of being practiced, carried out, and claimed in various ways.

### REFERENCES

The following references are cited within this specification. All of the cited references, including particularly any referenced patents and patent applications, are incorporated by reference in their entirety.
1. Fischetti, V. A., Nelson, D. & Schuch, R Reinventing phage therapy: are the parts greater than the sum? Nat Biotechnol 24, 1508-1511 (2006).
2. Andersson, D.1. & Hughes, D. Antibiotic resistance and its cost: is it possible to reverse resistance? Nat Rev Microbio/8, 260-271 (2010).
3. Woodford, N. & Ellington, M. J. The emergence of antibiotic resistance by mutation. Clin Microbiol Infect 13, 5-18 (2007).
4. Fenton, M., Ross, P., McAuliffe, 0., O'Mahony, J. & Coffey, A. Recombinant bacteriophage Iysins as antibacterials. Bioeng Bugs 1, 9-16.
5. Fischetti, V. A. Bacteriophage endolysins: a novel anti-infective to control Gram-positive pathogens. Int J Med Microbiol 300, 357-362.
6. Cheng, Q., Nelson, D., Zhu, S. & Fischetti, V. Removal of Group B Streptococci Colonizing the Vagina and Oropharynx of Mice with a Bacteriophage Lytic Enzyme. Antimicrobial Agents and Chemotherapy 49, 111-117 (2005).
7. Schuch, R, Nelson, D. & Fischetti, V. A. A bacteriolytic agent that detects and kills Bacillus anthracis. Nature 418, 884-889 (2002).
8. Fischetti, V. A. Bacteriophage endolysins: a novel anti-infective to control Gram-positive pathogens. tnt J Med Microbiol 300, 357-362 (2010).
9. Weidenmaier, C. & Peschel, A. Teichoic acids and related cell-wall glycopolymers in Gram-positive physiology and host interactions. Nat Rev Microbiol 6, 276-287 (2008).
10. Swoboda, J. G. et al. Discovery of a small molecule that blocks wall teichoic acid biosynthesis in Staphylococcus aureus. ACS Chem Bio/4, 875-883 (2009).
11. Schirner, K., Maries-Wright, J., Lewis, R J. & Errington, J. Distinct and essential morphogenic functions for wall- and lipo-teichoic acids in Bacillus subtilis. EMBO J. 28, 830-842 (2009).
12. Atilano, M. L. et al. Teichoic acids are temporal and spatial regulators of peptidoglycan cross-linking in Staphylococcus aureus. Proc Natl Acad Sci USA 107, 18991-18996 (2010).
13. Mayer-Scholl, A. et al. Human neutrophils kill Bacillus anthracis. PLoS Pathog 1, e23 (2005).
14. Grundling, A & Schneewind, O. Synthesis of glycerol phosphate lipoteichoic acid in Staphylococcus aureus. Proc Natl Acad Sci USA 104, 8478-8483 (2007).
15. Choudhury, B. et al. The structure of the major cell wall polysaccharide of Bacillus anthracis is species-specific. J Biol Chem 281, 27932-27941 (2006).
16. Helgason, E., Tourasse, N. J., Meisal, R., Caugant, D. A & Kolsto, A B. Multilocus sequence typing scheme for bacteria of the Bacillus cereus group. Appl Environ Microbiol 70, 191-201 (2004).
17. Leoff, C. et al. Cell wall carbohydrate compositions of strains from the Bacillus cereus group of species correlate with phylogenetic relatedness. J Bacteriol 190, 112-121 (2008).
18. Dubail, I. et al. Identification of an essential gene of Listeria monocytogenes involved in teichoic acid biogenesis. J Bacteriol 188, 6580-6591 (2006).
19. Soldo, B., Lazarevic, V., Pooley, H. M. & Karamata, D. Characterization of a Bacillus subtilis thermosensitive teichoic acid-deficient mutant: gene mnaA (yvyH) encodes the UDP-N-acetylglucosamine 2-epimerase. J Bacteriol 184, 4316-4320 (2002).
20. Velloso, L. M., Bhaskaran, S. S., Schuch, R., Fischetti, V. A & Stebbins, C. E. A structural basis for the allosteric regulation of non-hydrolysing UDP-GlcNAc 2-epimerases. EMBO Rep 9, 199-205 (2008).
21. Van Ert, M. N. et al. Global genetic population structure of Bacillus anthracis. PLoS ONE 2, e461 (2007).
22. Yoong, P., Schuch, R., Nelson, D. & Fischetti, V. A Identification of a broadly active phage lytic enzyme with lethal activity against antibiotic-resistant Enterococcus faecalis and Enterococcus faecium. Journal of Bacteriology 186, 4808-4812 (2004).
23. Palmer, K. L., Daniel, A, Hardy, C., Silverman, J. & Gilmore, M. S. Genetic basis for daptomycin resistance in enterococci. Antimicrob Agents Chemother 55, 3345-3356.
24. Brotz-Oesterhelt, H. & Sass, P. Postgenomic strategies in antibacterial drug discovery. Future Microbiol 5, 1553-1579.
25. Jorgensen, W. L. Efficient drug lead discovery and optimization. Acc Chem Res 42, 724-733 (2009).
26. Schuch, R. & Fischetti, V. A Detailed genomic analysis of the Wbeta and gamma phages infecting Bacillus anthracis: implications for evolution of environmental fitness and antibiotic resistance. J Bacterio/188, 3037-3051 (2006).
27. Ekwunife, F. S., Singh, J., Taylor, K. G. & Doyle, R. J. Isolation and Purification of CellWall Polysaccharide of Bacillus-Anthracis (Delta-Sterne). Ferns Microbiology Letters 82, 257-262 (1991).
28. Sabharwal, H. et al. Group A streptococcus (GAS) carbohydrate as an immunogen for protection against GAS infection. J Infect Dis 193, 129-135 (2006).
29. Fisher, N. & Hanna, P. Characterization of Bacillus anthracis germinant receptors in vitro. J Bacterio/187, 8055-8062 (2005).
30. Day, W. A, Rasmussen, S. L., Carpenter, B. M., Peterson, S. N. & Friedlander, A M. Microarray analysis of transposon insertion mutations in Bacillus anthracis: Global identification of genes required for sporulation and germination. Journal of Bacteriology 189, 3296-3301 (2007).
31. Raz, A & Fischetti, V. A Sortase A localizes to distinct foci on the Streptococcus pyogenes membrane. Proc Natl Acad Sci USA 105, 18549-18554 (2008).
32. Wiegand, I., Hilpert, K. & Hancock, R. E. Agar and broth dilution methods to determine the minimal inhibitory concentration (MIC) of antimicrobial substances. Nat Protoc 3, 163-175 (2008).
33. Bearss, U.S. patent application Ser. No. 12/454,062 (US 2009/0298900).
34. Samuel et al., Biochim Biophys Acta 1700(1):85-91 (2004).
35. Kawamura et al., J Biol Chem 254(17):8457-8465 (1979).
36. Kawamura et al., J Biol Chem 253(10):3595-3601 (1978).
37. Kuhn et al., FEMS Microbiol Rev 4(3):195-222, 58-464 (1988).
38. Hinderlich et al., J Biol Chem 272(39):24313-24318 (1997).
39. Keppler et al., Science 284(5418):1372-1376 (1999).
40. Ryan, P. A., Kirk, B. W., Euler, C. W., Schuch, R. & Fischetti, V. A. Novel algorithms reveal streptococcal transcriptomes and clues about undefined genes. PLoS Comput Biol 3, e132 (2007).
41. Schuch, R., Pelzek, A. J., Raz, A., Euler, C. W., Bhaskaran, S. S., Stebbins, C. E., Xu, Y., Clifford, A., Bearss, D. J., Vankayalapati, H., Goldberg, A. R., and Fischetti, V. A. Use of a bacteriophage lysin to identify a novel target for antimicrobial development. PLoS One, 2013, 8, e60754

We claim:

1. A method for treating a bacterial infection in a mammal, comprising administering to the mammal an effective amount of a composition comprising a pharmaceutically acceptable carrier and an inhibitor molecule that binds to the allosteric binding site of a bacterial 2-epimerase.

2. The method of claim 1, wherein the bacterial 2-epimerase is from a Gram-positive bacteria.

3. The method of claim 1, wherein the bacterial 2-epimerase is from a Gram-negative bacteria.

4. The method of claim 1, wherein the inhibitor molecule selectively binds to the bacterial 2-epimerase versus a mammalian 2-epimerase in a ratio of bacterial to mammalian 2-epimerase of at least 10:1.

5. The method of claim 1, wherein the inhibitor molecule selectively binds to the allosteric site versus the active site of the bacterial 2-epimerase in a ratio of allosteric to active site of at least 2:1.

6. The method of claim 1, wherein the enzymatic activity of the bacterial 2-epimerase is reduced or eliminated.

7. The method of claim 1, wherein the formation of bacterial wall material is disrupted, the bacterial cells are subject to dissolution, or a combination thereof.

8. The method of claim 1, wherein the inhibitor molecule exhibits contact points with at least 3 amino acid residues in the allosteric binding site of the bacterial 2-epimerase, wherein the amino acid residues comprise all or part of the twelve amino acid residues of an alignment consensus for the allosteric binding site of a plurality of bacterial 2-epimerases.

9. The method of claim 8, wherein the alignment consensus corresponds to at least four amino acid residues of the allosteric site of the bacterial 2-epimerase of SEQ ID NO. 1 selected from the group consisting of Q43, H44, Q46, M47, K67, R69, Q70, T102, E136, R210, E212 and H242.

10. The method of claim 8, wherein the inhibitor molecule exhibits contact points with at least 6-8 amino acid residues.

11. A composition for treating a bacterial infection in a mammal, comprising an effective amount of a composition comprising a pharmaceutically acceptable carrier and an inhibitor molecule that binds to the allosteric binding site of a bacterial 2-epimerase.

12. The composition of claim 11, wherein the bacterial 2-epimerase is from a Gram-positive bacteria.

13. The composition of claim 11, wherein the bacterial 2-epimerase is from a Gram-negative bacteria.

14. The composition of claim 11, wherein the inhibitor molecule selectively binds to the bacterial 2-epimerase versus a mammalian 2-epimerase in a ratio of bacterial to mammalian 2-epimerase of at least 10:1.

15. The composition of claim 11, wherein the inhibitor molecule selectively binds to the allosteric site versus the active site of the bacterial 2-epimerase in a ratio of allosteric to active site of at least 2:1.

16. The composition of claim 11, wherein the enzymatic activity of the bacterial 2-epimerase is reduced or eliminated.

17. The composition of claim 11, wherein the formation of bacterial wall material is disrupted, the bacterial cells are subject to dissolution, or a combination thereof.

18. The composition of claim 11, wherein the inhibitor molecule exhibits contact points with at least 3amino acid residues in the allosteric binding site of the bacterial 2-epimerase, wherein the amino acid residues comprise all or part of the twelve amino acid residues of an alignment consensus for the allosteric binding site of a plurality of bacterial 2-epimerases.

19. The composition of claim 18, wherein the alignment consensus corresponds to at least four amino acid residues of the allosteric site of the bacterial 2-epimerase of SEQ ID NO. 1 selected from the group consisting of Q43, H44, Q46, M47, K67, R69, Q70, T102, E136, R210, E212 and H242.

20. The composition of claim 18, wherein the inhibitor molecule exhibits contact points with at least 6-8 amino acid residues.

21. A method of evaluating binding affinities for inhibitors of bacterial growth, comprising the steps of:
a) conducting a computational modeling of an allosteric site in a bacterial 2-epimerase and a compound;
b) determining the number and type of contact points of the compound with amino acids within the allosteric site;
c) calculating a theoretical binding affinity of the compound in the allosteric site based on the number and character of the contact points; and
d) testing the compound in an assay to assess the modeling and theoretical binding affinity.

22. The method of claim 21, further comprising
e) creating a database of parameters to evaluate preferred contact points within the allosteric site.

23. The method of claim 21, wherein the compound is a compound of Formula I: 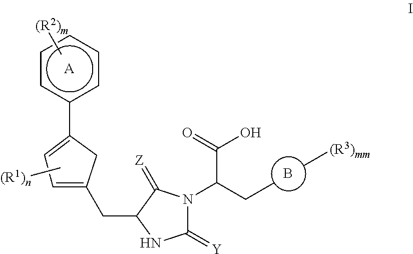
wherein X, Y, and Z each independently is O, S, or NR⁴; A is aryl or hetaryl; or A is halo; B is single-ringed aryl, hetaryl, or hetcyclyl; or B is CH₃; wherein A is halo and B is CH3 cannot occur in same compound; R¹ in each instance independently is C₀₋₄alkyl; R² in each instance independently is C₀₋₄alkyl, C₁₋₄ alkoxy, halo, —CF₂H, —CF₃, —OCF₃, —SCF₃, —SF₅; R³ in each instance independently is C₀₋₄ alkyl; R⁴ in each instance independently is C₀₋₄alkyl, or a single-ringed aryl, hetaryl, or hetcyclyl; n is 0, 1, or 2; and m and mm each independently is 0, 1, 2, 3, 4, or 5; or Formula II 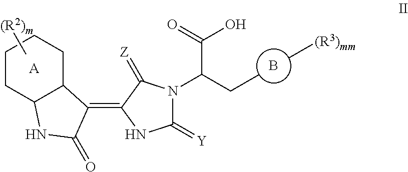
wherein Y, Z each independently is O, S, or NR⁴; A is aryl or hetaryl; B is single-ringed aryl, hetaryl, or hetcyclyl; R² in each instance independently is C₀₋₄ alkyl, C₁₋₄ alkoxy, halo, —CF₂H, —CF₃, —OCF₃, —SCF₃, —SF₅; R³ in each instance independently is C₀₋₄ alkyl; R⁴ in each instance independently is C₀₋₄ alkyl, or a single-ringed aryl, hetaryl, or hetcyclyl; n is 0, 1, or 2; and m and mm each independently is 0, 1, 2, 3, 4, or 5.

24. The method of claim 21, wherein the compound is selected from compounds 1-92 in Tables 7 and 8.

25. The method of claim 21, wherein the compound is 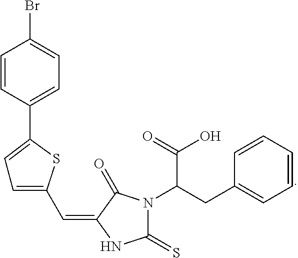 [SMILES: O=C(O)C(Cc1ccccc1)N1C(=O)/C(=C\c2ccc(-c3ccc(Br)cc3)s2)NC1=S]
